(19) United States
(12) Patent Application Publication
(10) Pub. No.: US 2024/0390286 A1
(43) Pub. Date: Nov. 28, 2024

(54) PREPARATION OF PHARMACEUTICAL COMPOSITIONS USING SUPERCYCLE VAPOR PHASE DEPOSITION OF METAL OXIDES

(71) Applicant: Applied Materials, Inc., Santa Clara, CA (US)

(72) Inventors: Miaojun Wang, Santa Clara, CA (US); Kai Zheng, Santa Clara, CA (US); Lance A. Scudder, Sunnyvale, CA (US); Sukti Chatterjee, San Jose, CA (US); Fei Wang, Fremont, CA (US); Shrikant Swaminathan, Santa Clara, CA (US); Pravin K. Narwankar, Sunnyvale, CA (US)

(21) Appl. No.: 18/674,730

(22) Filed: May 24, 2024

Related U.S. Application Data
(60) Provisional application No. 63/469,201, filed on May 26, 2023.

(51) Int. Cl. A61K 9/50

(52) U.S. Cl. CPC A61K 9/5089; A61K 9/501

(57) ABSTRACT

This disclosure pertains to methods to prepare coated particles comprising a drug-containing core and a coating of inorganic oxides applied by vapor phase deposition (supercycles). The coated particles have a modified drug release profile comparing to the uncoated drug particles.

### CROSS-REFERENCE TO RELATED APPLICATION

This application claims priority to U.S. Provisional Patent Application No. 63/469,201, filed May 26, 2023, the entire contents of which are incorporated herein by reference.

### TECHNICAL FIELD

This disclosure pertains to methods for preparing coated particles comprising a drug-containing core and a coating comprising an inorganic oxide (e.g., a metal oxide or metalloid oxide) and to coated particles having a coating that includes a layer composed of an inorganic oxide.

### BACKGROUND

It is of great interest to the pharmaceutical industry to develop improved formulations of drug substances. Formulation can influence the stability and bioavailability of the drug substance as well as other characteristics. Formulation can also influence various aspects of drug product manufacture, for example, the case and safety of the manufacturing process.

Controlled release formulations can have many benefits including: 1) Less frequent administration; 2) Reduced side effects; 3) Stable drug plasma concentration; and 4) Better patient adherence. While these advantages are significant, there are potential disadvantages to controlled release formulations, including possible toxicity or non-biocompatibility of the materials used to control release and undesirable by-products of degradation of the materials used to control release. It can be desirable for controlled release formulations to be inert, biocompatible, mechanically strong, capable of achieving high drug loading, safe from accidental release, simple to administer, and easy to make.

### SUMMARY

This disclosure pertains coated particles comprising a drug-containing core and an inorganic oxide coating (alternatively referred to as a coating layer) that completely encloses the particle. The coating can be applied by vapor phase deposition and can include one layer or multiple layers. A coating can be composed of two or more inorganic oxide layers that differ in composition. Thus, for example, one layer can be composed of aluminum, zinc and oxygen while another layer can be composed of zinc and oxygen. Together the two layers form the coating. The coating fully encloses the drug containing core and generally conforms to the core. The coated particles can have a modified drug release profile compared to the uncoated drug particles.

Vapor phase deposition methods for applying a coating to drug particles generally entail applying at least one layer of an inorganic oxide of a certain type (e.g., zinc oxide or silicon oxide) that is a few to several tens of nanometers thick. This is accomplished by carrying out, for example, 20-100 or more deposition cycles, wherein each cycle comprises sequential exposure to an inorganic oxide precursor and then an oxidant such as water or ozone (a precursor-oxidant pair) in which at least the precursor, and usually the oxidant, is the same in each cycle. For example, 20-100 cycles are carried out with trimethylaluminum and water as the precursor-oxidant pair. Such processes create an inorganic oxide layer that is composed of two elements (e.g., a layer composed essentially only of aluminum and oxygen). In some cases, the deposition of such an inorganic oxide layer, for example an aluminum oxide layer, is followed by deposition of a second, different inorganic oxide layer. For example, following deposition of an aluminum oxide layer, the coating process continues with 20-100 or more cycles using a different precursor-oxidant pair, for example, diethyl zinc and water. In this manner, two different inorganic oxide layers, each containing two elements, are created. Between the two layers there can exist a small interface region that is composed of three elements, in this example composed of aluminum, zinc and oxygen. However, this interface region in which three different elements are present is generally less than a nanometer thick.

In contrast to these earlier approaches for coating drug particles, the present methods entail carrying out a small number (e.g., 1, 2, 3, 4, 5, 6, 7, 8, 9 or 10) of cycles with a first precursor-oxidant pair, e.g., trimethylaluminum and water, and then carrying out a small number of cycles (e.g., 1, 2, 3, 4, 5, 6, 7, 8, 9 or 10) with a different precursor-oxidant pair (e.g., diethyl zinc and water) to create a layer containing three elements (e.g., aluminum, zinc and oxygen). The sequence of, for example, one cycle with a first precursor-oxidant pair and 3 cycles with second, different precursor-oxidant pair is called a “supercycle” and can be repeated a number of times, for example, 2-10 times, 5-20 times or 2-50 or more times. The entire coating can be composed of such a layer. However, the process can continue by using vapor phase deposition to apply a conventional, two element inorganic oxide layer, e.g., a zinc oxide layer. Instead, the process can continue with a second set of supercycles each of which can have a different number of cycles using the first precursor-oxidant pair and the second precursor-oxidant pairs (e.g., 2 cycles with the first precursor-oxidant pair and 10 cycles with second precursor-oxidant pair). Carried out in this specific manner, the process created two supercycle layers adjacent to each other that both contain the same three elements (e.g., aluminum, zinc and oxygen), but in differing proportions. In another variation, the process can continue with a second set of supercycles using different precursor-oxidant pairs. When two different precursor-oxidant pairs are used, the oxidants can be the same or different.

When carried out with two different precursor-oxidant pairs (e.g., trimethylaluminum/water and diethyl zinc/water) that differ in the metal or metalloid, the supercycle process creates a relatively uniform layer primarily composed of three elements (e.g., aluminum, zinc and oxygen (“AZO”)). This can be considered essentially a ternary oxide. When the supercycle process is carried out to create a layer, the layer can include small regions composed of two elements (e.g., aluminum and oxygen or zinc and oxygen). For example, when more zinc oxide precursor is used, there may be a small region composed of zinc oxide. However, overall, the layer would still be composed of three elements. The method permits fine tuning of the characteristics of the coating. This is, at least in part, due to the ability to combine aspects of two different inorganic oxides such as aluminum oxide and zinc oxide. For example, an aluminum oxide layer is generally relatively dense and amorphous and can provide a barrier that is relatively less permeable to, for example, water. In contrast, a zinc oxide layer is generally relatively crystalline and has grain boundaries that make the coating relatively porous compared to, for example, an aluminum oxide coating. Combining these properties has the advantage of creating a layer that is relatively less permeable to water but has a relatively small amount of aluminum, which can be desirable in certain circumstances.

As another example, the supercycle process can be used to create a relatively uniform layer that primarily of composed of aluminum, silicon and oxygen (“ASO”) and, sometimes, regions compound of aluminum and oxygen or silicon and oxygen. For example, when more silicon oxide precursor is used, there may be a small region composed of silicon oxide. However, overall the layer would still be composed of three elements. An ASO layer can be created by carrying out just a few (e.g., 1, 2, 3, 4, 5, 6, 7, 8, 9 or 10) cycles with a first precursor-oxidant pair, e.g., trimethylaluminum and water, and then carrying out a number of cycles (e.g., 1, 2, 3, 4, 5, 6, 7, 8, 9 or 10) with a different precursor-oxidant pair (e.g., SiCl₄ and water). The process can then continue with a second set of supercycles each of which can have a different number of cycles using the first precursor-oxidant pair and the second precursor-oxidant pairs (e.g., 2 cycles with the first precursor-oxidant pair and 10 cycles with second precursor oxidant pair). When carried out with two different precursor-oxidant pairs (e.g., trimethylaluminum/water and SiCl₄/water), this process creates a coating that has a relatively high proportion of ternary compound composed of three elements (e.g., aluminum, silicon and oxygen (ASO)) and a relatively small proportion of binary compound composed of two elements (e.g., aluminum and oxygen or silicon and oxygen).

This process described herein is referred herein as a “supercycle vapor phase deposition process” or simply a “supercycle” process. When such a process is carried out with a combination of aluminum oxide precursor and zinc oxide precursor, the resulting layer coating is referred to as an aluminum/zinc oxide (AZO) layer. When such a process is carried out with a combination of aluminum oxide precursor and a silicon oxide precursor, the resulting layer is referred to as an aluminum/silicon oxide (ASO) layer. When such a process is carried out with a combination of zinc oxide precursor and a silicon oxide precursor, the resulting layer is referred to as an aluminum/silicon oxide (ZSO) layer. A layer created only with an aluminum oxide precursor and an oxident is called an aluminum oxide layer or “AlOx” layer. A layer created only with a zinc oxide precursor and an oxidant is called a zinc oxide layer or “ZnOx” coating. A layer created only with a silicon oxide precursor and an oxidant is called a silicon oxide coating or “SiOx” layer.

As shown in the examples, the supercycle vapor phase deposition process can be used to coat drug-containing particles to provide a slower drug dissolution rate compared to uncoated drug particles. Importantly by, for example, altering the ratio of cycles using an aluminum oxide precursor to the number of cycles using a zinc oxide precursor or silicon oxide precursor and/or altering the total number of each type of cycle it is possible to fine tune coating composition and thus the release properties (the dissolution rate of the drug contained in the particle) or other properties of the coated particle.

In one aspect, the disclosure is related to a method of preparing coated particles comprising drug-containing core enclosed by an inorganic oxide coating, wherein the drug-containing core comprises a drug, the method comprising the sequential steps of:
(a) loading particles comprising a drug into a chamber of a reactor (e.g., particles having a median particle size, on a volume average basis between 0.1 μm and 500,000 μm, 200,000 μm, 100,000 μm, 50,000 μm, 10,000 μm 1,000 μm, 100 μm 50 μm, or 10 μm);
(b) performing a first number of first cycles, wherein each first cycle comprises steps (b1)-(b4):
(b1) applying a vaporous or gaseous first inorganic oxide precursor to the particles in the reactor by pulsing the vaporous or gaseous first inorganic oxide precursor into the reactor;
(b2) purging using an inert gas or performing one or more pump-purge cycles of the reactor using an inert gas;
(b3) applying a vaporous or gaseous oxidant to the particles in the reactor by pulsing the oxidant into the reactor;
(b4) purging using an inert gas or performing one or more pump-purge cycles of the reactor using an inert gas;
(c) performing a second number of second cycles, wherein each second cycle comprises steps (c1)-(c4):
(c1) applying a vaporous or gaseous second inorganic oxide precursor to the particles in the reactor by pulsing the vaporous or gaseous second inorganic oxide precursor into the reactor;
(c2) purging using an inert gas or performing one or more pump-purge cycles of the reactor using an inert gas;
(c3) applying a vaporous or gaseous oxidant to the particles in the reactor by pulsing the oxidant into the reactor;
(c4) purging using an inert gas or performing one or more pump-purge cycles of the reactor using an inert gas; and
(d) repeating steps (b)-(c) at least once;
   wherein, the first inorganic oxide precursor and second inorganic oxide precursor are different (i.e., the metal or metalloid differ between two); in each repeat of step (b) the first number is independently selected from 1-10 or 1-20; and in each repeat of step (c) the second number is independently selected from 1-10 or 1-20.

In some embodiments, the first inorganic oxide precursor is an aluminum oxide precursor and the second inorganic oxide precursor is a zinc oxide precursor.

In some embodiments, the first inorganic oxide precursor is a zinc oxide precursor and the second inorganic oxide precursor is an aluminum oxide precursor.

In some embodiments, the first inorganic oxide precursor is an aluminum oxide precursor and the second inorganic oxide precursor is a silicon oxide precursor.

In some embodiments, the first inorganic oxide precursor is a silicon oxide precursor and the second inorganic oxide precursor is an aluminum oxide precursor.

In some embodiments, the first inorganic oxide precursor is a zinc oxide precursor and the second inorganic oxide precursor is a silicon oxide precursor.

In some embodiments, the first inorganic oxide precursor is a zinc oxide precursor and the second inorganic oxide precursor is an aluminum oxide precursor.

In some embodiments, the first inorganic oxide precursor is a titanium oxide precursor and the second inorganic oxide precursor is an aluminum oxide precursor.

In some embodiments, the first inorganic oxide precursor is a titanium oxide precursor and the second inorganic oxide precursor is a zinc oxide precursor.

In some embodiments, the first inorganic oxide precursor is a titanium oxide precursor and the second inorganic oxide precursor is a silicon oxide precursor.

In some embodiments, the first inorganic oxide precursor is a aluminum oxide precursor and the second inorganic oxide precursor is a titanium oxide precursor.

In some embodiments, the first inorganic oxide precursor is a zinc oxide precursor and the second inorganic oxide precursor is a titanium oxide precursor.

In some embodiments, the first inorganic oxide precursor is a silicon oxide precursor and the second inorganic oxide precursor is a titanium oxide precursor.

In some embodiments, the aluminum oxide precursor is trimethylaluminum (TMA).

In some embodiments, the zinc oxide precursor is diethylzinc (DEZ).

In some embodiments, the silicon oxide precursor is SiCl₄, Tris(tertpentoxy) silanol, diisopropylamino silane (DIPAS) or 1,2-Bis(diisopropylamino)disilane (BDIPADS).

In some embodiments, the inorganic oxide coating layer constitutes 1-20% wt/wt of the coated particles.

In some embodiments, each of the first and second inorganic oxide precursors are selected from DEZ and TMA and either: a) the first inorganic oxide precursor is TMA and the second inorganic oxide precursor is DEZ; or b) the first inorganic oxide precursor is DEZ and the second inorganic oxide precursor is TMA.

In some embodiments, the first number is 1, 2, 3, 4, or 5 and the second number is between 1 and 10.

In some embodiments, the first number is 1.

In some embodiments, the second number is 1, 2, 3, 4 or 5, 2-10, 2-15, 2-20, 2-30, 2-40.

In some embodiments, the second number is 3.

In some embodiments, the second number is 4.

In some embodiments, steps (b)-(c) occur 1-5, 1-10, 1-15, 5-10, 5-15, 10-20, 10-30, 5-40, 5-50, 5-100 times.

In some embodiments, each of steps (b1), (b3), (c1) and (c3) comprises: (i) introducing the vaporous or gaseous inorganic oxide precursor into the chamber, (ii) allowing a holding time to pass, and (iii) pumping the vaporous or gaseous inorganic oxide precursor of the chamber; and repeating steps (i)-(ii) at least once.

In some embodiments, some or all of the residual vaporous or gaseous first inorganic oxide precursor is pumped out of the reactor prior to step (b3).

In some embodiments, some or all of the residual vaporous or gaseous oxidant is pumped out of the reactor prior to step (c).

In some embodiments, some or all of the residual vaporous or gaseous second inorganic oxide precursor is pumped out of the reactor prior to step (c3).

In some embodiments, the first cycles and second cycles take place at a temperature between 25° C. and 60° C.

In some embodiments, the oxidant in step (b3) is water.

In some embodiments, the oxidant in step (c3) is water.

In some embodiments, step (a) further comprises agitating the particles.

In some embodiments, each pump-purge cycle comprises flowing the inert gas into the reactor chamber to a desired pressure. This can displace residual reactants and by-products.

In some embodiments, each pump-purge cycle comprises flowing the inert gas into the reactor chamber to a desired pressure and after a delay time pumping the inert gas out of the reactor until the pressure of the inert gas is below 1 torr and repeating the steps of flowing the inert gas into the reactor chamber to a desired pressure and after a delay time pumping the inert gas out of the reactor until the pressure of the inert gas is below 1 torr.

In some embodiments, the method further comprises agitating the particles in the reactor throughout steps (a)-(d).

In some embodiments, the particles are not removed from the reactor during steps (a)-(d).

In some embodiments, the method further comprises, after step (d):
(e) performing a third number of third cycles, wherein each third cycle comprises steps (e1)-(e4):
   (e1) applying a vaporous or gaseous third inorganic oxide precursor to the particles in the reactor by pulsing the vaporous or gaseous third precursor into the reactor;
   (e2) purging using an inert gas or performing one or more pump-purge cycles of the reactor using an inert gas;
   (e3) applying a vaporous or gaseous oxidant to the particles in the reactor by pulsing the oxidant into the reactor;
   (e4) purging using an inert gas or performing one or more pump-purge cycles of the reactor using an inert gas;
(f) performing a fourth number of fourth cycles, wherein each fourth cycle comprises steps (f1)-(f4):
   (f1) applying a vaporous or gaseous fourth inorganic oxide precursor to the particles in the reactor by pulsing the vaporous or gaseous fourth precursor into the reactor;
   (f2) purging using an inert gas or performing one or more pump-purge cycles of the reactor using an inert gas;
   (f3) applying a vaporous or gaseous oxidant to the particles in the reactor by pulsing the oxidant into the reactor;
   (f4) purging using an inert gas or performing one or more pump-purge cycles of the reactor using an inert gas; and
(g) repeating steps (e)-(f) at least once.
   wherein, the third and fourth precursor are different, and the third number is selected from 1-10, and the fourth number is selected from 1-10.

In some embodiments, the first and third or first and fourth precursors are an aluminum precursor and the second and fourth or first and third precursors are a zinc precursor.

In some embodiments, the first and third precursors or first and fourth precursors are an zinc precursor and the second and fourth or second and third precursors are an aluminum precursor.

In some embodiments, the first and third or first and fourth precursors are a zinc precursor and the second and fourth or first and third precursors are an aluminum precursor.

In some embodiments, the first and third precursors or first and fourth precursors are an aluminum precursor and the second and fourth or second and third precursors are an zinc precursor.

In some embodiments, the aluminum precursor is trimethylaluminum (TMA).

In some embodiments, the zinc precursor is diethylzinc (DEZ).

In some embodiments, the coating constitutes 1-20% wt/wt of the coated particles.

In some embodiments, steps (e)-(f) occur 1-40 times.

In some embodiments, the third number is 1 or 2 or 1-10 and the fourth number is between 1 and 10.

In some embodiments, the third number is 1.

In some embodiments, the fourth number is 2, 3, 4, −5, 2-10, 2-15, 2-20, 2-30, 2-40.

In one aspect, the disclosure is related to a coated particle prepared by the method described herein.

In some embodiments, the coated particle has a slower drug release compared to the uncoated drug particles.

In one aspect, the disclosure is related to a coated particle comprising a core comprising a drug enclosed by an inorganic oxide coating comprising a uniform layer composed of three elements (e.g., aluminum, zinc and oxygen; aluminum, silicon and oxygen; or zinc, aluminum and oxygen).

In one aspect, the disclosure is related to a coated particle comprising a core comprising a drug enclosed by an inorganic oxide coating comprising a uniform layer composed of three elements (e.g., aluminum, zinc and oxygen; aluminum, silicon and oxygen; or zinc, aluminum and oxygen) and a uniform layer composed of two elements (e.g., aluminum and oxygen; silicon and oxygen; or and oxygen).

In one aspect, the disclosure is related to a coated particle comprising a core comprising a drug enclosed by an inorganic oxide coating comprising a two different uniform layers composed of three elements (e.g., aluminum, zinc and oxygen; aluminum, silicon and oxygen; or zinc, aluminum and oxygen) and the two different layers differ with respect to the three elements (e.g., one is composed of aluminum, zinc and oxygen and the other is composed of aluminum, silicon and oxygen) or in the proportion of the three elements (e.g., one layer is composed of aluminum, zinc and oxygen with a Al/Zn ratio greater than 1; and the other layer is also composed of aluminum, zinc and oxygen with a Al/Zn ratio less than 1).

In some embodiments, the layer composed of three elements is at least 2 nm thick.

In some embodiments, the layer composed of three elements is 2-50 nm thick.

In some embodiments, the coated drug particle is 5-30% by weight inorganic oxide.

In some embodiments, the weight ratio of aluminum to zinc in the inorganic oxide coating is between 0.01 and 0.8.

In some embodiments, the inorganic oxide coating comprises a layer composed of two elements (e.g., zinc and oxygen, aluminum and oxygen or zinc and oxygen) in addition to a layer composed of three elements.

In some embodiments, the layer composed of two elements or three elements is at least 1 nm thick.

In some embodiments, the layer composed of two elements or three elements is 1-50 nm thick.

In some embodiments, the core consists of an drug.

In some embodiments, the core has a D50 on a volume average basis of 100 nm-30 micrometers.

In one aspect, the disclosure is related to a pharmaceutical composition comprising the coated particle described herein and a pharmaceutically acceptable excipient or carrier.

In one aspect, the disclosure is related to a pharmaceutical composition comprising the coated particle described herein and water.

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a composition, “about” may mean+/−10% of the recited value. For instance, a composition including about 100 ng/ml of a given compound may include 90˜110 ng/ml of the compound.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

FIG. 1 depicts a schematic illustration of an exemplary reactor system.

FIG. 2 depicts a schematic illustration of exemplary coated particles.

FIG. 3 depicts a schematic illustration of an exemplary supercycle. The exemplary supercycle contains n number of AlOx cycles and m number of ZnOx cycles. The AlOx cycle includes applying a vaporous or gaseous aluminum precursor (e.g., trimethylaluminum or TMA); performing one or more pump-purge cycles of the reactor using an inert gas; applying a vaporous or gaseous oxidant (e.g., H₂O) to the particles in the reactor by pulsing the oxidant into the reactor; and performing one or more pump-purge cycles of the reactor using an inert gas. The ZnOx cycle includes applying a vaporous or gaseous zinc precursor (e.g., diethylzinc or DEZ); performing one or more pump-purge cycles of the reactor using an inert gas; applying a vaporous or gaseous oxidant (e.g., H₂O) to the particles in the reactor by pulsing the oxidant into the reactor; and performing one or more pump-purge cycles of the reactor using an inert gas.

FIG. 4 depicts a schematic illustration of an exemplary cycle in a supercycle. The cycle contains applying a vaporous or gaseous precursor (e.g., TMA or DEZ); performing one or more pump-purge cycles of the reactor using an inert gas; applying a vaporous or gaseous oxidant (e.g., H₂O) to the particles in the reactor by pulsing the oxidant into the reactor; and performing one or more pump-purge cycles of the reactor using an inert gas.

FIG. 5 depicts a schematic illustration of particle coated with multiple layers. In this example, an AlOx layer is applied using conventional cycles, then an AZO layer is applied using supercycles. Finally a ZnOx layer is applied using conventional cycles. The Al/Zn ratio in the AZO layer can be adjusted by varying the numbers of AlOx cycles and ZnOx cycles. The overall coating thickness and the coating wt % can be adjusted by varying the number of conventional cycles and the number of supercycles.

FIG. 6A depicts the results of XRD analysis before and after an AlOx, ZnOx, or AZO layer was applied to particles of indomethacin using a supercycle coating process for AZO or conventional cycles for AlOx and ZnOx. As shown in the figure, there was no significant change in XRD signals after the coating process, indicating that there was no structural change in the indomethacin after the coating process, and that the indomethacin was not damaged by the coating process.

FIG. 6B depicts the results of FTIR analysis before and after an AlOx, ZnOx, or AZO coating was applied to particles of indomethacin using a supercycle coating process (for AZO). As shown in the figure, there was no significant change in FTIR signals after the coating process, indicating that there was no chemical change in the indomethacin after the coating process, and that the indomethacin was not damaged by the coating process.

FIG. 7 depicts the results of a study of indomethacin dissolution in pH 7.2 sodium phosphate buffer solution in a USP2 dissolution apparatus (paddle) at 37° C. at 100 rpm stirring speed. As shown in the figure, both uncoated particles and particles coated with ZnOx exhibited an immediate indomethacin release profile. The AlOx coated particles exhibited minimal indomethacin release. By comparison, the AZO coated particles exhibited controlled indomethacin release with the release % controlled by AZO oxide composition (Al/Zn ratio). Again, the indomethacin release rates for AZO coated particles were between that of ZnOx coated particles and that of AlOx coated particles. The indomethacin release rate of AZO coated particles decreases as the Al/Zn ratio increases.

FIG. 8 depicts the results of a study of indomethacin dissolution in pH 7.2 PIPES buffer solution in a USP4 dissolution apparatus (flow through) at 37° C. at a 16 ml/min flow rate. As shown in the figure, uncoated particles exhibited an immediate indomethacin release profile. The AlOx coated particles exhibited minimal indomethacin release. By comparison, the AZO coated particles exhibited controlled indomethacin release with release % controlled by AZO oxide composition (Al/Zn ratio). Again, the indomethacin release rates for AZO coated particles were between that of ZnOx coated particles and that of AlOx coated particles. The release rate of AZO coated particles decreases as the Al/Zn ratio increases.

FIG. 9 depicts the result of a study of indomethacin dissolution in pH 7.2 sodium phosphate buffer solution in a USP2 dissolution apparatus (paddle) at 37° C. at 100 rpm stirring speed. As shown in the figure, uncoated particles exhibited an immediate indomethacin release profile. By comparison, the coated particles exhibited controlled indomethacin release.

FIG. 10 depicts the results of a study or indomethacin dissolution in pH 7.2 sodium phosphate buffer solution in a USP2 dissolution apparatus (paddle) at 37° C. at 100 rpm stirring speed. As shown in the figure, uncoated particles exhibited an immediate indomethacin release profile. By comparison, the AlOx coated particles exhibited controlled indomethacin release. The release rate of the AlOx coated particles decreased as the thickness of the AlOx coating increases.

FIGS. 11A-11C are TEM images of coated indomethacin particles. FIG. 11A is a cross-section of an AlOx coated indomethacin particle. FIG. 11B is a cross-section of an AZO coated indomethacin particle described in Table 1. FIG. 11C is a cross-section of ZnOx coated indomethacin particle.

FIGS. 12A-12C are TEM images of coated indomethacin particles. FIG. 12A is a cross-section of a coated indomethacin particle having an AlOx inner layer and an AZO outer layer. FIG. 12B is a cross-section of a coated indomethacin particle having an ZnOx inner layer and an AlOx outer layer. FIG. 12C is a cross-section of a coated indomethacin particle having an AZO coating with supercycle process starting with DEZ first. A zinc rich layer is observed at the interface between the indomethacin and the coating.

FIGS. 13A-13F are SEM images of the uncoated and coated indomethacin particles. FIGS. 13A-13C are SEM images of uncoated particles. FIGS. 13D-13F are SEM images of coated particles. No significant change in morphology was observed after the AZO coating process.

FIG. 14A-14B shows the TEM/EELS elemental mapping of an AZO coating. FIG. 14B shows the line scan data. In FIG. 14A, the first panel on the left depicts the TEM image of the area being mapped, and the other panels depict the EELS elemental mapping results of C, Al, Zn, O, Cl, and N. The results show relatively homogeneous distribution of Al and Zn across the thickness of the AZO coating.

### DETAILED DESCRIPTION

This disclosure pertains to methods for preparing coated particles comprising a drug-containing core and a coating comprising an inorganic oxide. The coating can be applied by vapor phase deposition. The coated particles have a modified drug release profile comparing to the uncoated drug particles.

### Drug

The drugs or drug substances (i.e., active pharmaceutical ingredients) that can be coated are organic compounds selected from the group that includes: an analgesic, an anesthetic, an anti-inflammatory agent, an anthelmintic, an anti-arrhythmic agent, an antiasthma agent, an antibiotic, an anticancer agent, an anticoagulant, an antidepressant, an antidiabetic agent, an antiepileptic, an antihistamine, an antitussive, an antihypertensive agent, an antimuscarinic agent, an antimycobacterial agent, an antineoplastic agent, an antioxidant agent, an antipyretic, an immunosuppressant, an immunostimulant, an antithyroid agent, an antiviral agent, an anxiolytic sedative, a hypnotic, a neuroleptic, an astringent, a bacteriostatic agent, a beta-adrenoceptor blocking agent, a blood product, a blood substitute, a bronchodilator, a buffering agent, a cardiac inotropic agent, a chemotherapeutic, a contrast media, a corticosteroid, a cough suppressant, an expectorant, a mucolytic, a diuretic, a dopaminergic, an antiparkinsonian agent, a free radical scavenging agent, a growth factor, a haemostatic, an immunological agent, a lipid regulating agent, a muscle relaxant, a parasympathomimetic, a parathyroid calcitonin, a biphosphonate, a prostaglandin, a radio-pharmaceutical, a hormone, a sex hormone, an anti-allergic agent, an appetite stimulant, an anoretic, a steroid, a sympathomimetic, a thyroid agent, a vaccine, a vasodilator and a xanthinc.

In some embodiments, the drug is in the form of uncoated particles. In some embodiments, uncoated particles have a surface area by BET (Brunauer, Emmett and Teller) specific surface area of more than 0.1 m²/g, more than 0.2 m²/g, more than 0.5 m²/g, more than 1 m²/g, more than 2 m²/g, more than 5 m²/g, or more than 10 m²/g. In some embodiments, the uncoated particles have a surface area by BET of less than 0.1 m²/g, less than 0.2 m²/g, less than 0.5 m²/g, less than 1 m²/g, less than 2 m²/g, less than 5 m²/g, or less than 10 m²/g. In some embodiments, the uncoated particles have a surface area by BET of 0.1-10 m²/g, 0.2-10 m²/g, 0.5-10 m²/g, 1-10 m²/g, 2-10 m²/g, or 5-10 m²/g. In some embodiments, the uncoated particles have a surface area by BET of about 2 m²/g.

In some embodiments, the uncoated particles have a D10 of less than 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D10 of more than 0.1 μm, more than 0.2 μm, more than 0.5 μm, more than 1 μm, more than 2 μm, more than 5 μm, more than 10 μm, on a volume average basis. In some embodiments, the uncoated particles have a D10 of 0.1 μm to 1 μm, 0.1 μm to 10 μm, or 0.1 μm to 20 μm on a volume average basis. In some embodiments, the uncoated particles have a D10 of about 1 μm on a volume average basis.

In some embodiments, the uncoated particles have a D50 of greater than about 0.1 μm, less than 0.2 μm, less than 0.5 μm, less than 1 μm, less than 2 μm, less than 5 μm, less than 10 μm, less than 20 μm, or less than 50 μm, on a volume average basis. In some embodiments, the uncoated particles have a D50 of more than 1 μm, more than 2 μm, more than 5 μm, more than 10 μm, more than 20 μm, or more than 50 μm, on a volume average basis. In some embodiments, the uncoated particles have a D50 of 1 μm to 500 μm, 1 μm to 100 μm, 1 μm to 10 μm, or 1 μm to 50 μm on a volume average basis. In some embodiments, the uncoated particles have a D50 of about 5 μm on a volume average basis.

In some embodiments, the uncoated particles have a D90 of less than 10 μm, less than 20 μm, or less than 50 μm, less than 200 μm, on a volume average basis. In some embodiments, the uncoated particles have a D90 of more than 10 μm, more than 20 μm, or more than 50 μm, on a volume average basis. In some embodiments, the coated particles have a D90 of 200 μm to 2000 μm on a volume average basis. In some embodiments, the uncoated particles have a D90 of 10 μm to 200 μm, 10 μm to 20 μm, or 10 μm to 50 μm on a volume average basis. In some embodiments, the uncoated particles have a D90 of about 20 μm on a volume average basis.

### Vapor Phase Deposition

The coatings are applied by vapor phase deposition using a precursor molecule and an oxidant (e.g., ozone or water vapor). Vapor phase deposition of inorganic oxides is sometimes referred to as atomic layer deposition (ALD). However, depending on a number of factors, including the surface being coated, each cycle of the deposition reaction does not necessarily deposit one atomic layer on the entire surface.

### Reactor System

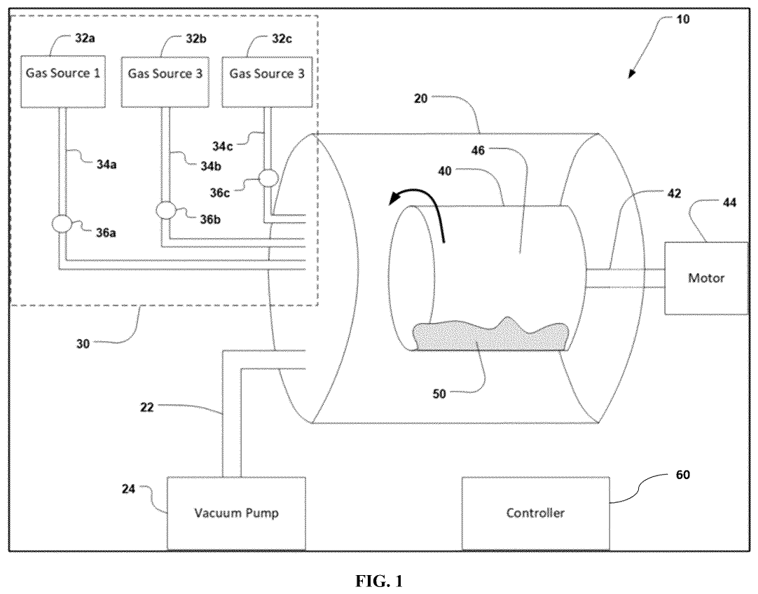

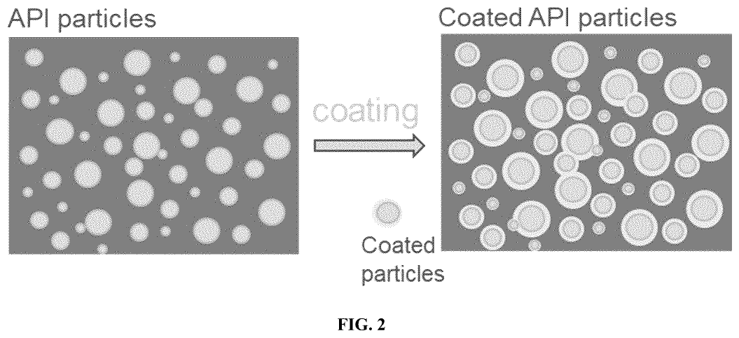

The term “reactor system” in its broadest sense includes all systems that could be used to perform vapor phase deposition or atomic layer deposition. An exemplary reactor system is illustrated in FIG. 1 and further described below.

The reactor system 10 can perform vapor phase deposition or atomic layer deposition. The reactor system 10 permits the process to be performed at higher (above 50° C., e.g., 50-100° C. or higher) or lower process temperature, e.g., below 50° C., e.g., at or below 25° C. For example, the reactor system 10 can form thin-film inorganic oxide on the particles primarily at temperatures of 40-80° C., e.g., 40° C. or 80° C. In general, the particles can remain or be maintained at such temperatures. This can be achieved by having the reactants and/or the interior surfaces of the reactor chamber (e.g., the chamber 20 and drum 40 discussed below) remain or be maintained at such temperatures.

Again, illustrating a vapor phase deposition or atomic layer deposition process, the reactor system 10 includes a stationary vacuum chamber 20 which is coupled to a vacuum pump 24 by vacuum tubing 22. The vacuum pump 24 can be an industrial vacuum pump sufficient to establish pressures less than 1 Torr, e.g., 1 to 100 mTorr, e.g., 50 mTorr. The vacuum pump 24 permits the chamber 20 to be maintained at a desired pressure and permits removal of reaction byproducts and unreacted process gases.

In operation, the reactor 10 performs the vapor phase deposition or atomic layer deposition process by introducing a gaseous oxidant and aluminum (or zinc) precursor into the chamber 20. The gaseous oxidant and aluminum (or zinc) precursor are introduced alternatively into the reactor. In addition, the reaction can be performed at low temperature conditions, such as below 80° C., e.g., below 50° C., below 30° C., or below 25° C. In some embodiments, the operating temperature is 50° C. In some embodiments, the operating temperature is above 5° C., above 10° C., above 15° C., above 20° C., above 25° C., above 30° C., above 35° C., above 40° C., above 45° C., above 50° C., above 56° C., above 60° C., above 65° C., above 70° C., above 75° C., or above 80° C. In some embodiments, the operating temperature is below 20° C., below 25° C., below 30° C., below 35° C., below 40° C., below 45° C., below 50° C., below 56° C., below 60° C., below 65° C., below 70° C., below 75° C., or below 80° C.

The chamber 20 is also coupled to a chemical delivery system 30. The chemical delivery system 30 includes three or more gas sources 32a, 32b, 32c coupled by respective delivery lines 34a, 34b, 34c and controllable valves 36a, 36b, 36c to the vacuum chamber 20. The chemical delivery system 30 can include a combination of restrictors, gas flow controllers, pressure transducers, and ultrasonic flow meters to provide controllable flow rate of the various gasses into the chamber 20. The chemical delivery system 30 can also include one or more temperature control components, e.g., a heat exchanger, resistive heater, heat lamp, etc., to heat or cool the various gasses before they flow into the chamber 20. Although FIG. 1 illustrates separate gas lines extending in parallel to the chamber for each gas source, two or more of the gas lines could be joined, e.g., by one or more three-way valves, before the combined line reaches the chamber 20.

One of the gas sources can provide an oxidant. In particular, a gas source can provide a vaporous or gaseous oxidant. For example, the oxidant can be ozone. As another example, the oxidant can be water vapor.

One of the gas sources can be an aluminum (or zinc) precursor. In particular, a gas source can provide a vaporous or gaseous aluminum (or zinc) precursor. For example, the aluminum precursor can be TMA.

One of the gas sources can provide a purge gas. In particular, the third gas source can provide a gas that is chemically inert to the oxidant and aluminum (or zinc) precursor, the coating, and the particles being processed. For example, the purge gas can be N₂, or a noble gas, such as argon.

A rotatable coating drum 40 is held inside the chamber 20. The drum 40 can be connected by a drive shaft 42 that extends through a sealed port in a side wall of the chamber 20 to a motor 44. The motor 44 can rotate the drum at speeds of 1 to 100 rpm. Alternatively, the drum can be directly connected to a vacuum source through a rotary union.

The particles to be coated, shown as a particle bed 50, are placed in an interior volume 46 of the drum 40. The drum 40 and chamber 20 can include scalable ports (not illustrated) to permit the particles to be placed into and removed from the drum 40.

The body of the drum 40 is provided by one or more of a porous material, a solid metal, and a perforated metal. The pores through the cylindrical side walls of the drum 40 can have a dimension of 1-10 μm.

In operation, one of the gasses flows into chamber 20 from the chemical delivery system 30 as the drum 40 rotates. A combination of pores (1-100 μm), holes (0.1-10 mm), or large openings in the coating drum 40 serve to confine the particles in the coating drum 40 while allowing rapid delivery of precursor chemistry and the pumping of byproducts or unreacted species. Due to the pores in the drum 40, the gas can flow between the exterior of the drum 40, i.e., the reactor chamber 20, and the interior of the drum 40. In addition, rotation of the drum 40 agitates the particles to expose new surfaces of the powder bed, ensuring a large surface area of the particles remains exposed to the process gas. This permits fast, uniform interaction of the particle surface with the process gas.

In some implementations, one or more temperature control components are integrated into the drum 40 to permit control of the temperature of the drum 40. For example, a resistive heater, a thermoelectric cooler, or other component can be in or on the side walls of the drum 40.

The reactor system 10 also includes a controller 60 coupled to the various controllable components, e.g., vacuum pump 24, gas distribution system 30, motor 44, a temperature control system, etc., to control operation of the reactor system 10. The controller 60 can also be coupled to various sensors, e.g., pressure sensors, flow meters, etc., to provide closed loop control of the pressure of the gasses in the chamber 20.

In general, the controller 60 can operate the reactor system 10 in accord with a “recipe.” The recipe specifies an operating value for each controllable element as a function of time. For example, the recipe can specify the times during which the vacuum pump 24 is to operate, the times of and flow rate for each gas source 32a, 32b, 32c, the rotation rate of the motor 44, etc. The controller 60 can receive the recipe as computer-readable data (e.g., that is stored on a non-transitory computer readable medium).

The controller 60 and other computing device parts of systems described herein can be implemented in digital electronic circuitry, or in computer software, firmware, or hardware. For example, the controller can include a processor to execute a computer program as stored in a computer program product, e.g., in a non-transitory machine-readable storage medium. Such a computer program (also known as a program, software, software application, or code) can be written in any form of programming language, including compiled or interpreted languages, and it can be deployed in any form, including as a standalone program or as a module, component, subroutine, or other unit suitable for use in a computing environment. In some implementations, the controller 60 is a general-purpose programmable computer. In some implementations, the controller can be implemented using special purpose logic circuitry, e.g., an FPGA (field programmable gate array) or an ASIC (application specific integrated circuit).

### Operation

Initially, particles are loaded into the drum 40 in the reactor system 10. The particles can be purely particles of a drug (or a combination of particles of a first drug and a second drug) or a mixture of particles of a drug (or a combination of particles of a first drug and a second drug) and particles of an excipient. In some cases, the particles are composed of one or more drugs (e.g., one of the drugs discussed above) and one or more excipients. Once any access ports are sealed, the controller 60 operates the reactor system 10 according to the recipe in order to form the thin-film aluminum (or zinc) oxide on the particles.

In particular, the oxidant and the aluminum (or zinc) precursor can be alternately supplied to the chamber 20, with each step of supplying an oxidant or the aluminum (or zinc) precursor followed by a purge cycle in which the inert gas is supplied to the chamber 20 to force out the excessive oxidant or aluminum (or zinc) precursor and by-products used in the prior step. Moreover, one or more of the gases (aluminum (or zinc) precursor gases and/or the inert gas and/or oxidant gas) can be supplied in pulses in which the chamber 20 is filled with the gas to a specified pressure, a holding time is permitted to pass, and the chamber is evacuated by the vacuum pump 24 before the next pulse commences.

In particular, the controller 60 can operate the reactor system 10 as follows.

In an aluminum (or zinc) precursor half-cycle, while the motor 44 rotates the drum 40 to agitate the particles 50:
i) The gas delivery system 30 is operated to flow the aluminum (or zinc) precursor gas, e.g., trimethylaluminum (TMA) or diethylzinc (DEZ)), from the source 32a into the chamber 20 until a first specified pressure is achieved. The specified pressure can be 0.1 Torr to half of the saturation pressure of the aluminum (or zinc) precursor gas (e.g., 0.3-2 torr).
ii) Flow of the aluminum (or zinc) precursor is halted, and a specified holding time (e.g., 60 seconds) is permitted to pass, e.g., as measured by a timer in the controller. This permits the aluminum (or zinc) precursor to flow through the particle bed in the drum 40 and react with the surface of the particles 50 inside the drum 40.
iii) The vacuum pump 50 evacuates the chamber 20, e.g., down to pressures below 1 Torr, e.g., to 1 to 100 mTorr, e.g., 50 mTorr.

Next, in a first purge cycle, while the motor 44 rotates the drum to agitate the particles 50:
iv) The gas delivery system 30 is operated to flow the inert gas, e.g., N₂, from the source 32c into the chamber 20 until a second specified pressure is achieved. The second specified pressure can be 1 to 100 Torr.
v) Flow of the inert gas is halted, and a specified delay time is permitted to pass, e.g., as measured by the timer in the controller. This permits the inert gas to flow through the pores in the drum 40 and diffuse through the particles 50 to displace the aluminum (or zinc) precursor gas and any vaporous by-products.
vi) The vacuum pump 50 evacuates the chamber 20, e.g., down to pressures below 1 Torr, e.g., to 1 to 500 mTorr, e.g., 50 mTorr.

These steps (iv)-(vi) can be repeated a number of times set by the recipe, e.g., six to twenty times, e.g., sixteen times.

In an oxidant half-cycle, while the motor 44 rotates the drum 40 to agitate the particles 50:
vii) The gas delivery system 30 is operated to flow the oxidant (e.g., water or ozone), from the source 32a into the chamber 20 until a third specified pressure is achieved. The third pressure can be 0.1 Torr to half of the saturation pressure of the oxidant gas (e.g., 2-8 torr).
viii) Flow of the oxidant is halted, and a specified holding time is permitted to pass, e.g., as measured by the timer in the controller. This permits the oxidant to flow through the pores in the drum 40 and react with the surface of the particles 50 inside the drum 40.
ix) The vacuum pump 50 evacuates the chamber 20, e.g., down to pressures below 1 Torr, e.g., to 1 to 500 mTorr, e.g., 50 mTorr.

Next, a second purge cycle is performed. This second purge cycle can be identical to the first purge cycle, or can have a different number of repetitions of the steps (iv)-(vi) and/or different delay time and/or different pressure.

The cycle of the aluminum (or zinc) precursor half-cycle, first purge cycle, oxidant half cycle and second purge cycle can be repeated a number of times set by the recipe, e.g., one to ten times.

As noted above, the coating process can be performed at a low process temperature, e.g., below 80° C., e.g., at or below 50° C., at or below 35° C., or at or below 25° C. In some embodiments, the operating temperature is 50° C. In some embodiments, the operating temperature is above 5° C., above 10° C., above 15° C., above 20° C., above 25° C., above 30° C., above 35° C., above 40° C., above 45° C., above 50° C., above 56° C., above 60° C., above 65° C., above 70° C., above 75° C., or above 80° C. (e.g. 20° C. to 80° C.). In some embodiments, the operating temperature is below 20° C., below 25° C., below 30° C., below 35° C., below 40° C., below 45° C., below 50° C., below 56° C., below 60° C., below 65° C., below 70° C., below 75° C., or below 80° C. In particular, the particles can remain or be maintained at such temperatures during all of steps (i)-(ix) noted above. In general, the temperature of the interior of the reactor chamber does not exceed 80° C. during of steps (i)-(ix). This can be achieved by having the oxidant gas, aluminum (or zinc) precursor gas and inert gas be injected into the chamber at such temperatures during the respective cycles. In addition, physical components of the chamber can remain or be maintained at such temperatures, e.g., using a cooling system, e.g., a thermoelectric cooler, if necessary.

### Methods for Preparing a Coated Particle by Supercycles

In one aspect, the disclosure provides methods to prepare a coated particle that has a drug-containing core an inorganic oxide coating layer applied using supercycles, each supercycle having a first number of first cycles and a second number of second cycles. In some embodiments, the coated particle has a modified drug release profile compared to the uncoated drug particles.

The methods include the sequential steps of: (a) providing particles comprising a drug; (b) performing a first number of first cycles; using first inorganic oxide precursor and (c) performing a second number of second cycles using a second inorganic oxide precursor, wherein the first and second inorganic oxide precursors are for forming different inorganic oxides (e.g., the first precursor can be aluminum oxide precursor and the second precursor can be zinc oxide precursor). The vaporous or gaseous oxidant used in the first and second cycles can be the same or different.

The step of performing a first number of first cycles (step (b)) comprises: (b1) applying a vaporous or gaseous first inorganic oxide precursor to the particles in the reactor by pulsing the vaporous or gaseous first inorganic oxide precursor into the reactor; (b2) purging using an inert gas or performing one or more pump-purge cycles of the reactor using an inert gas; (b3) applying a vaporous or gaseous oxidant to the particles in the reactor by pulsing the oxidant into the reactor; and (b4) purging using an inert gas or performing one or more pump-purge cycles of the reactor using an inert gas.

The step of performing a second number of second cycles (step (c)) comprises: (c1) applying a vaporous or gaseous second inorganic oxide precursor to the particles in the reactor by pulsing the vaporous or gaseous second inorganic oxide precursor into the reactor; (c2) purging using an inert gas or performing one or more pump-purge cycles of the reactor using an inert gas; (c3) applying a vaporous or gaseous oxidant to the particles in the reactor by pulsing the oxidant into the reactor; and (c4) purging using an inert gas or performing one or more pump-purge cycles of the reactor using an inert gas.

The steps (b)-(c) constitutes a supercycle. In some embodiments, steps (b)-(c) are performed two or more times to increase the total thickness of the coating. In some embodiments, the particles are agitated prior to and/or during step (a). In some embodiments, the reactor pressure is allowed to stabilize following step (b1), step (b2), step (b3) and/or step (b4). In some embodiments, the reactor pressure is allowed to stabilize following step (c1), step (c2), step (c3) and/or step (c4).

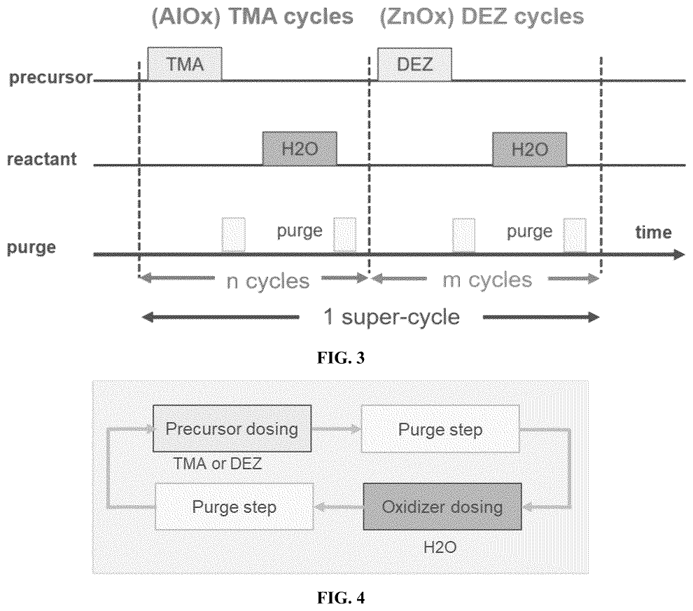

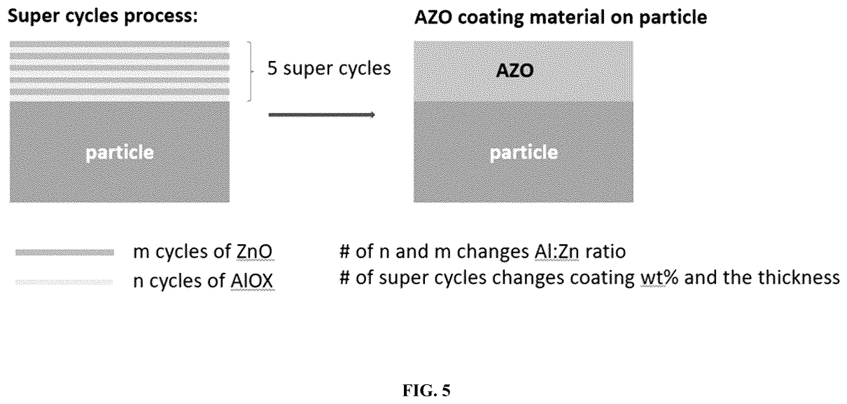

In some embodiments, aluminum oxide and zinc oxide precursors are applied using supercycles to create an AZO layer (see, e.g., FIG. 3). In some embodiments, the aluminum precursor is trimethylaluminum (TMA). In some embodiments, the zinc precursor is diethylzinc (DEZ). In some embodiments, the zinc precursor is zinc tetrachloride.

In some embodiments, aluminum oxide and silicon oxide precursors are applied using supercycles to create an ASO layer. In some embodiments, the aluminum precursor is TMA. In some embodiments, the silicon precursor is SiCl₄, Tris(tertpentoxy) silanol, diisopropylamino silane (DIPAS) or 1,2-Bis(diisopropylamino)disilane (BDIPADS).

A supercycle includes a first number of first cycles (e.g., TMA cycles) and a second number of second cycles (e.g., DEZ cycles). In some embodiments, the aluminum/zinc (Al/Zn) ratio in the coating can be adjusted by varying the number of first cycles (e.g., TMA cycles) and the number of second cycles (e.g., DEZ cycles). In some embodiments, the first number is selected from 1-10, and the second number is selected from 1-10. In some embodiments, the first number is 1, 2, 3, 4, 5, 6, 7, 8, 9, 10, 11, 12, 13, 14, 15, 16, 17, 18, 19, 20, 21, 22, 23, 24, 25, 30, 40, or 50. In some embodiments, the second number is 1, 2, 3, 4, 5, 6, 7, 8, 9, 10, 11, 12, 13, 14, 15, 16, 17, 18, 19, 20, 21, 22, 23, 24, 25, 30, 40, or 50. In some embodiments, the first number is 1 and the second number is selected from 1-5. In some embodiments, the first number is 1 and the second number is 2. In some embodiments, the first number is 1 and the second number is 3. In some embodiments, the first number is 1 and the second number is 4.

In some embodiments, multiple supercycles are used to create a AZO layer or ASO layer. In some embodiments, the number of supercycles is more than 1, more than 2, more than 3, more than 4, more than 5, more than 6, more than 7, more than 8, more than 9, more than 10, more than 15, more than 20, more than 25, more than 30, more than 35, more than 40, more than 45, more than 50, more than 60, more than 70, more than 80, more than 90, more than 100, more than 110, more than 120, more than 130, more than 140, or more than 150. In some embodiments, the number of supercycles is less than 2, less than 3, less than 4, less than 5, less than 6, less than 7, less than 8, less than 9, less than 10, less than 15, less than 20, less than 25, less than 30, less than 35, less than 40, less than 45, less than 50, less than 60, less than 70, less than 80, less than 90, less than 100, less than 110, less than 120, less than 130, less than 140, or less than 150. In some embodiments, the number of supercycles is 5-50, 5-25, or 5-10.

In some embodiments, multiple first supercycles with a first Al/Zn ratio are followed by multiple second supercycles with a second Al/Zn ratio to deposit an AZO layer composed of layers with two or more different Al/Zn ratios.

In some embodiments, multiple supercycles are used to create an ASO layer. In some embodiments, multiple first supercycles with a first Al/Si ratio are followed by multiple second supercycles with a second Al/Si ratio to deposit an ASO layer composed of layers with two or more different Al/Si ratios or a gradient Al/Si ratio.

In some embodiments, 5-10 supercycles are performed wherein the first number is 1 and the second number is 3. In some embodiments, 5-25 additional supercycles are performed wherein the first number is 1 and the second number is 2.

In some embodiments, 5-10 additional supercycles are performed wherein the first number is 1 and the second number is 4. In some embodiments, 5-25 additional supercycles are performed wherein the first number is 1 and the second number is 3.

In some embodiments there are multiple different layers. For example an AZO layer can be combined with AlOx layer and/or ZnOx layer to provide various different coating structures. The AZO layer can be an inner layer and the AlOx or ZnOx layer can be an outer layer. In some embodiments, exemplary coating structures include an AlOx layer and an AZO layer or an ZnOx layer and an AZO layer.

In some embodiments there are multiple different layers. For example, an ASO layer can be combined with AlOx layer and/or SiOx layer to provide various different coating structures. The ASO layer can be an inner layer and the AlOx or SiOx layer can be an outer layer. In some embodiments, exemplary coating structures include an AlOx layer and an ASO layer or an SiOx layer and an ASO layer.

In some embodiments, the aluminum/zinc (Al/Zn) ratio (wt/wt) in the coated particles is more than 0.01, more than 0.02, more than 0.03, more than 0.04, more than 0.05, more than 0.06, more than 0.07, more than 0.08, more than 0.09, more than 0.1, more than 0.11, more than 0.12, more than 0.13, more than 0.14, more than 0.15, more than 0.2, more than 0.25, more than 0.3, more than 0.35, more than 0.4, more than 0.45, more than 0.5, more than 0.6, more than 0.7, more than 0.9, or more than 0.9. In some embodiments, the aluminum/zinc (Al/Zn) ratio in the coated particles is less than 0.01, less than 0.02, less than 0.03, less than 0.04, less than 0.05, less than 0.06, less than 0.07, less than 0.08, less than 0.09, less than 0.1, less than 0.11, less than 0.12, less than 0.13, less than 0.14, less than 0.15, less than 0.2, less than 0.25, less than 0.3, less than 0.35, less than 0.4, less than 0.45, less than 0.5, less than 0.6, less than 0.7, less than 0.9, or less than 0.9. In some embodiments, the Al/Zn ratio in the coated particles is 0.1-0.5, 0.1-0.4, 0.1-0.3, 0.15-0.3 or 0.15-0.35. In some embodiments, the Al/Zn ratio in the coated particles is less than 0.7. In some embodiments, the Al/Zn ratio in the coated particles is 0.1-0.7.

In some embodiments, the entirety of the coating has a thickness in the range of 0.1 nm to 100 nm, 0.1 nm to 50 nm, 0.1 nm to 10 nm, 0.1 to 5 nm, 1 nm to 50 nm, 1 nm to 10 nm, or 1 nm to 5 nm. In some embodiments, the entirety of the coating has a thickness of more than 0.1 nm, more than 0.2 nm, more than 0.3 nm, more than 0.4 nm, more than 0.5 nm, more than 0.6 nm, more than 0.7 nm, more than 0.8 nm, more than 0.9 nm, more than 1 nm, more than 2 nm, more than 3 nm, more than 4 nm, more than 5 nm, more than 6 nm, more than 7 nm, more than 8 nm, more than 9 nm, more than 10 nm, more than 15 nm, more than 20 nm, more than 30 nm, more than 40 nm, more than 50 nm, or more than 100 nm. In some embodiments, the entirety of the coating has a thickness of less than 0.1 nm, less than 0.2 nm, less than 0.3 nm, less than 0.4 nm, less than 0.5 nm, less than 0.6 nm, less than 0.7 nm, less than 0.8 nm, less than 0.9 nm, less than 1 nm, less than 2 nm, less than 3 nm, less than 4 nm, less than 5 nm, less than 6 nm, less than 7 nm, less than 8 nm, less than 9 nm, less than 10 nm, less than 15 nm, less than 20 nm, less than 30 nm, less than 40 nm, less than 50 nm, or less than 100 nm. In some embodiments, the entirety of the coating has a thickness of between 10 nm and 50 nm. In some embodiments, the entirety of the coating has a thickness of between 10 nm and 200 nm, between 10 nm and 100 nm, between 10 nm and 50 nm, or between 25 nm and 50 nm. In some embodiments, the entirety of the coating has a thickness of 10-60 nm, 10-50 nm, 10-40 nm or 10-30 nm.

In some cases an individual layer in a multi-layer coating has thickness in the range of 0.1 nm to 100 nm, 0.1 nm to 50 nm, 0.1 nm to 10 nm, 0.1 to 5 nm, 1 nm to 50 nm, 1 nm to 10 nm, or 1 nm to 5 nm. In some embodiments, the aluminum oxide layer has a thickness of more than 0.1 nm, more than 0.2 nm, more than 0.3 nm, more than 0.4 nm, more than 0.5 nm, more than 0.6 nm, more than 0.7 nm, more than 0.8 nm, more than 0.9 nm, more than 1 nm, more than 2 nm, more than 3 nm, more than 4 nm, more than 5 nm, more than 6 nm, more than 7 nm, more than 8 nm, more than 9 nm, more than 10 nm, more than 15 nm, more than 20 nm, more than 30 nm, more than 40 nm, more than 50 nm, or more than 100 nm. In some embodiments, the coating has a thickness of less than 0.1 nm, less than 0.2 nm, less than 0.3 nm, less than 0.4 nm, less than 0.5 nm, less than 0.6 nm, less than 0.7 nm, less than 0.8 nm, less than 0.9 nm, less than 1 nm, less than 2 nm, less than 3 nm, less than 4 nm, less than 5 nm, less than 6 nm, less than 7 nm, less than 8 nm, less than 9 nm, less than 10 nm, less than 15 nm, less than 20 nm, less than 30 nm, less than 40 nm, less than 50 nm, or less than 100 nm. In some embodiments, the coating has a thickness of between 1 nm and 30 nm. In some embodiments, the coating has a thickness of between 1 nm and 20 nm. In some embodiments, the coating has a thickness of 2-5 nm, 5-10 nm, or 10-20 nm. In some embodiments, individual AZO, ASO, ZSO, ZnOx, AlOx or SiOx layers can have a thickness of 1-10 nm, 5-10 nm, 5-20 nm or 10-20 nm.

In some embodiment, the layer composed of three elements has a Al/Zn, Al/Si or Al/Zn that gradually changes across the layer from the inner side to the outer side. This can be accomplished by adjust the ration of the two different precursors as the layer with 3 compounds is being deposited.

### Coated Particles

In some embodiments, the disclosure provides coated particles comprising a drug-containing core and an AZO or ASO layer.

In some embodiments, the coated particles have an improved flowability compared to uncoated drug particles.

In some embodiments, the coating layer includes a supercyle coating layer that is an AZO or ASO coating layer.

In some embodiments, the particle comprises a layer that is composed of Al, Zn and O, and the Al/Zn ratio varies within the layer.

In some embodiments, the particle comprises a layer that is composed of Al, SI and O, and the Al/Si ratio varies within the layer.

In some embodiments, the particle comprises a layer that is composed of Si, Zn and O, and the Si/Zn ratio varies within the layer.

In some embodiments, AZO or ASO is present throughout the entire thickness of the coating.

In some embodiments, the supercycle coating layer is amorphous.

In some embodiments, the supercycle coating comprises aluminum oxide and zinc oxide. In some embodiments, the supercycle coating comprises a homogenous layer.

In some embodiments, both aluminum oxide and zinc oxide are distributed throughout the supercycle coating layer.

In some embodiments, the structure of the drug can be assessed by X-Ray Diffraction (XRD) analysis. In some embodiments, there are no significant changes in XRD signals before and after the coating process. In some embodiments, there is no structural change in the drug after the coating process, and that the drug was not damaged by the coating process.

In some embodiments, the structure of the drug can be assessed by Fourier-transform infrared (FTIR) analysis. In some embodiments, there are no significant changes in FTIR signals before and after the coating process. In some embodiments, there is no chemical change in the drug after the coating process, and that the drug was not damaged by the coating process.

In some embodiments, the composition of the coated particles can be assessed by Thermogravimetric Analysis (TGA) analysis. In some embodiments, the amount of inorganic component constitutes more than 0.1%, more than 0.2%, more than 0.3%, more than 0.4%, more than 0.5%, more than 0.6%, more than 0.7%, more than 0.8%, more than 0.9%, more than 1%, more than 1.2%, more than 1.4%, more than 1.6%, more than 1.8%, more than 2%, more than 2.2%, more than 2.4%, more than 2.6%, more than 2.8%, more than 3%, more than 3.2%, more than 3.4%, more than 3.6%, more than 3.8%, more than 4%, more than 4.2%, more than 4.4%, more than 4.6%, more than 4.8%, more than 5%, more than 6%, more than 7%, more than 8%, more than 9%, more than 10%, more than 12%, more than 14%, more than 16%, more than 18%, or more than 20% wt/wt of the coated particles.

In some embodiments, weight percent of inorganic oxide in the coated particles is less than 0.1%, less than 0.2%, less than 0.3%, less than 0.4%, less than 0.5%, less than 0.6%, less than 0.7%, less than 0.8%, less than 0.9%, less than 1%, less than 1.2%, less than 1.4%, less than 1.6%, less than 1.8%, less than 2%, less than 2.2%, less than 2.4%, less than 2.6%, less than 2.8%, less than 3%, less than 3.2%, less than 3.4%, less than 3.6%, less than 3.8%, less than 4%, less than 4.2%, less than 4.4%, less than 4.6%, less than 4.8%, less than 5%, less than 6%, less than 7%, less than 8%, less than 9%, less than 10%, less than 12%, less than 14%, less than 16%, less than 18%, or less than 20% wt/wt of the coated particles. In some embodiments, the weight percent of inorganic oxide in the coated particles is 0.1%-20%, 0.5%-10%, 1%-10%, 1%-5%, 2%-5%, 1%-4%, 1%-3%, or 2%-4% wt/wt of the coated particles. In some embodiments, the amount of inorganic component constitutes about 1%-20% wt/wt of the coated particles.

In some embodiments, the dissolution or drug release of the coated particles can be assessed by an in vitro release over time (dissolution) analysis. In some embodiments, the dissolution or drug release of the coated particles can be assessed by HPLC analysis. In some embodiments, the dissolution is assessed in methanol. In some embodiments, the dissolution is assessed in a sodium phosphate buffer solution (PBS) (e.g., pH 7.2, with or without surfactant) at 37° C., with a stirring of 100 revolutions per minute (RPM), for more than 1 minute, more than 2 minutes, more than 5 minutes, more than 10 minutes, more than 20 minutes, more than 30 minutes, more than 40 minutes, more than 50 minutes, more than 60 minutes, more than 120 minutes, more than 3 hours, more than 4 hours, more than 5 hours, more than 6 hours, more than 7 hours, more than 8 hours, more than 12 hours, more than 16 hours, more than 24 hours. In some embodiments, the coated particles have a reduced dissolution rate compared to uncoated drug particles. In some embodiments, the dissolution rate of the coated particles is at least more than 5%, more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500%, or more than 600%, lower than the dissolution of uncoated particles (drug-containing cores). In some embodiments, uncoated particles exhibited an immediate release profile. In some embodiments, AlOx coated particles showed minimal release. In some embodiments, the AZO coated particles showed controlled release. In some embodiments, the release rates for AZO coated particles were in between that of ZnOx coated particles and that of AlOx coated particles. In some embodiments, the release rate of AZO coated particles decreases as the Al/Zn ratio increases. In some embodiments, the release rate of AZO coated particles can be adjusted by varying the Al/Zn ratio. In some embodiments, the release rate of AZO coated particles can be adjusted by varying the thickness of the coating. In some embodiments, the release rate of ASO coated particles can be adjusted by varying the Al/Si ratio. In some embodiments, the release rate of ASO coated particles can be adjusted by varying the thickness of the coating.

In some embodiments, the coated particles have a zinc oxide outer layer at the outer surface. In some embodiments, the coated particles have an aluminum oxide outer layer.

In some embodiments, the coated particles have a hydrophilic outer surface. In some embodiments, the coated particles have 30-90% drug release in 30 minutes. In some embodiments, the coated particles have a slow drug release rate. In some embodiments, the coated particles are dispersed in PBS.

In some embodiments, the coated particles have more than 10%, more than 20%, more than 30%, more than 40%, more than 50%, more than 60%, more than 70%, more than 80%, more than 90%, more than 95% drug release in 30 minutes. the coated particles have less than 10%, less than 20%, less than 30%, less than 40%, less than 50%, less than 60%, less than 70%, less than 80%, less than 90%, less than 95% in 30 minutes. In some embodiments, the coated particles have 100% drug release in 30 minutes.

In some embodiments, comparing to uncoated particles (drug-containing core), the coated particles have at least more than 10%, more than 20%, more than 30%, more than 40%, more than 50%, more than 60%, more than 70%, more than 80%, more than 90%, more than 95% slower drug release.

In some embodiments, the uncoated particles (drug-containing core) have an immediate release (e.g., about 90-100% drug release in 30 minutes). In some embodiments, the coated particles have a controlled release (e.g., about 1-10% release in 30 minutes).

In some embodiments, the particles (drug-containing core) are also coated with aluminum oxide layer and/or a zinc oxide layer in addition to the AZO coating layer. For example, the aluminum oxide layer can be 2-50 nm (e.g., 5-20 nm, 5-10 nm) thick and the zinc oxide layer can be 2-50 nm (e.g., 5-20 nm, 5-10 nm) thick.

In some embodiments, the morphology of the coated particles can be assessed by Transmission Electron Microscopy (TEM) analysis. In some embodiments, there is no obvious change in particle size before and after the coating process. In some embodiments, there is no obvious morphology change in the drug before and after the coating process.

In some embodiments, the morphology of the coated particles can be assessed by Scanning Electron Microscopy (SEM) analysis. In some embodiments, there is no obvious change in particle size before and after the coating process. In some embodiments, there is no obvious morphology change in the drug before and after the coating process.

### Pharmaceutical Compositions

Also provided herein are pharmaceutical compositions that contain the coated particles. The pharmaceutical compositions can be formulated in any suitable manner known in the art. In some embodiments, the pharmaceutical compositions can be in the form of tablets, capsules, powders, microparticles, granules, syrups, suspensions, solutions, nasal spray, transdermal patches, injectable solutions, or suppositories.

Pharmaceutical compositions are formulated to be compatible with their intended route of administration (e.g., oral, intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents (e.g., benzyl alcohol or methyl parabens, chlorobutanol, phenol, ascorbic acid, and thimerosal), antioxidants (e.g., ascorbic acid and sodium bisulfite), chelating agents (e.g., ethylenediaminetetraacetic acid), buffers (e.g., acetates, citrates, and phosphates), and isotonic agents (e.g., sugars (e.g., dextrose), polyalcohols (e.g., mannitol or sorbitol), and salts (e.g., sodium chloride)), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating (e.g., lecithin) or a surfactant. Controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Pharmaceutically acceptable carriers, adjuvants and vehicles that can be used in the pharmaceutical compositions of the present disclosure include ion exchangers, alumina, aluminum stearate, lecithin, serum proteins (e.g., human serum albumin), buffer substances (e.g., phosphates, glycine), sorbic acid, potassium sorbate, partial glyceride mixtures of saturated vegetable fatty acids, water, salts or electrolytes (e.g., protamine sulfate, disodium hydrogen phosphate, potassium hydrogen phosphate, sodium chloride, and zinc salts), colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms can contain the coated particles described herein in the range of 0.001% to 100% (e.g., 0.1-95%, 20-80%, or 75-85%) with the balance made up from the suitable pharmaceutically acceptable excipients.

In some embodiments, the supercycle coating can simply the formulation process or other manufacturing process of a pharmaceutical composition. In some embodiments, the supercycle coating eliminates the need to include additional detergents in the final formulation.

### EXAMPLES

### Example 1: AZO Coating with Different Compositions (Different Al/Zn Ratios) Using Supercycle Processes

A supercycle process was used to coat particles of indomethacin. Each supercycle includes (1) applying a number of aluminum oxide cycles; (2) applying a number of zinc oxide cycles.

Examples of the supercycle sequences used in this example include:
Supercycle 1 (SC1): (TMA/H₂O)×1+(DEZ/H₂O)×1;
Supercycle 2 (SC2): (TMA/H₂O)×1+(DEZ/H₂O)×2;
Supercycle 3 (SC3): (TMA/H₂O)×1+(DEZ/H₂O)×3;
Supercycle 4 (SC4): (TMA/H₂O)×1+(DEZ/H₂O)×4;
Supercycle 5 (SC5): (TMA/H₂O)×1+(DEZ/H₂O)×8
Supercycle 6 (SC6): (TMA/H₂O)×1+(DEZ/H₂O)×7

Control AlOx-coated and ZnOx-coated particles were also created. In general, the operating temperature was 50° C.; the TMA, DEZ and H₂O pressures in the process were 2 Torr; and the purging nitrogen pressure was 8 torr. Table 1, below, describes certain characteristics of the coating indomethacin particles used in the studies described below. The AZO-1, AZO-2, AZO-3 and AZO-4 samples have an AZO coating applied using a supercycle process. AlOx sample has a coating applied using conventional cycles with TMA and water. ZnOx sample has a coating applied using conventional cycles with DEZ and water.

To evaluate the properties of the coated particles, the coated particles were subjected to various analyses.

Thermogravimetric Analysis (TGA) analysis

Table 1 shows the TGA analysis results of the coated particles. As shown, the total inorganic material (oxide wt %) constitutes about 10-18% of the coated particles.

### Inductively Coupled Plasma Optical Emission Spectroscopy (ICP-OES) Analysis

Table 1 also shows the ICP-OES analysis results of the coated particles. As shown, the total inorganic material (oxide wt %) constitutes about 10-18% of the coated particles.

### Al/Zn Ratio Analysis

Table 1 also shows the weight ratio of aluminum to zinc in the coated particles, as determined by ICP-OES. As shown, the Al/Zn ratio was about 0.10-0.69.

TABLE 1
Analysis of Oxide Coating

| | Al/Zn | Oxide wt % | Oxide wt % |
|---|---|---|---|
| Sample | (wt ratio) | (ICP-OES) | (TGA) |
| AlOx-coated | 100/0 | n/a | 13.0 |
| AZO-1 coated | 0.69 | 11.0 | 10.3 |
| AZO-2 coated | 0.31 | 16.2 | 16.7 |
| AZO-3 coated | 0.18 | 17.4 | 17.6 |
| AZO-4 coated | 0.096 | 15.3 | 15.3 |
| ZnOx-coated | 0 | 16.3 | 16.4 |

### Transmission Electron Microscopy (TEM) Analysis

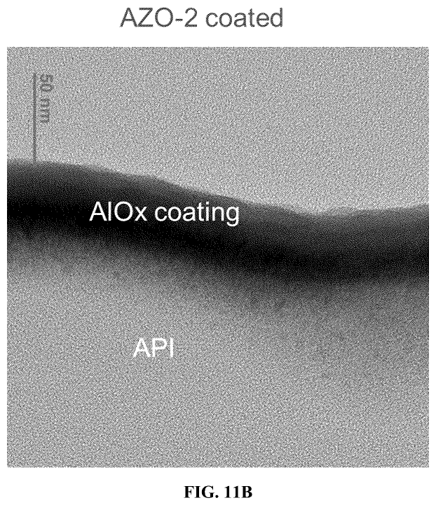

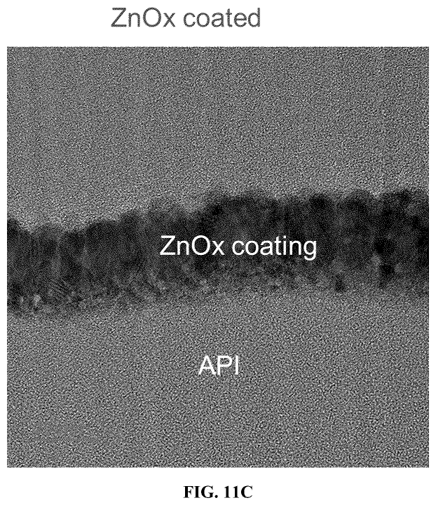

FIGS. 11A-11C show the TEM images of the coated particles.

FIG. 11A shows a TEM image of an AlOx coated particle.

FIG. 11B shows a TEM image of an AZO-2 coated particle.

FIG. 11C shows a TEM image of a ZnOx coated particle.

As shown in FIGS. 11A-11C, the AlOx and AZO-2 coatings were amorphous, while the ZnOx coating was polycrystalline.

### Scanning Electron Microscopy (SEM) Analysis

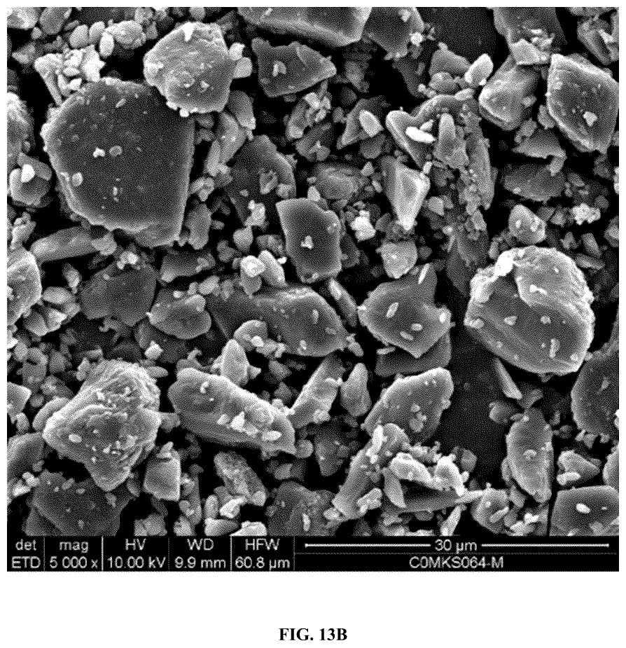

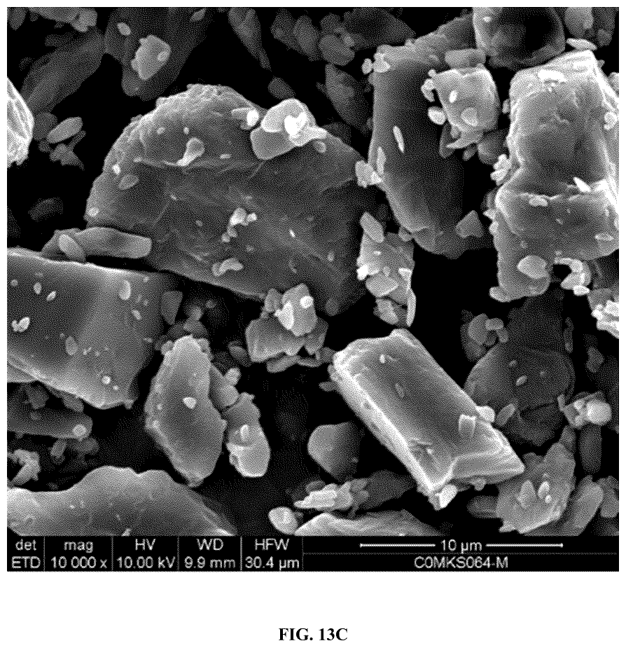

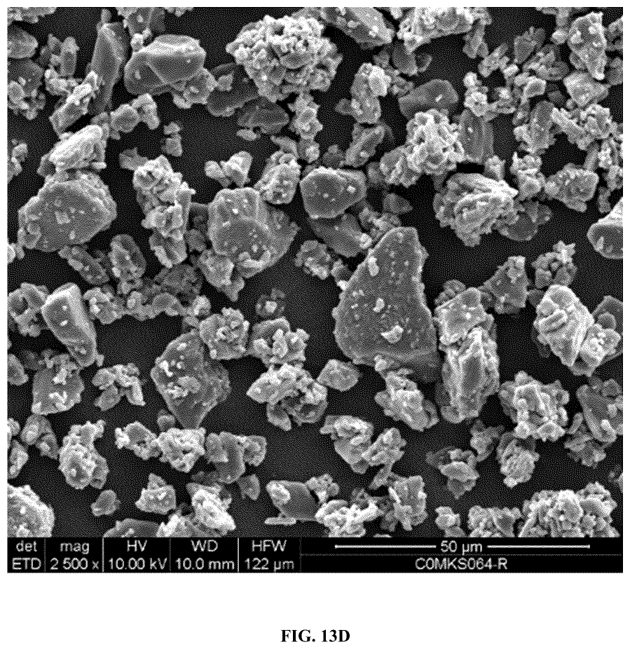

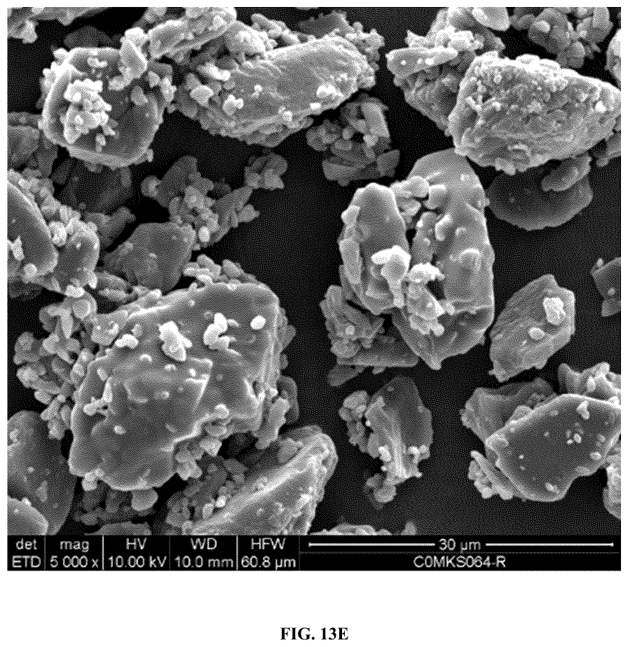

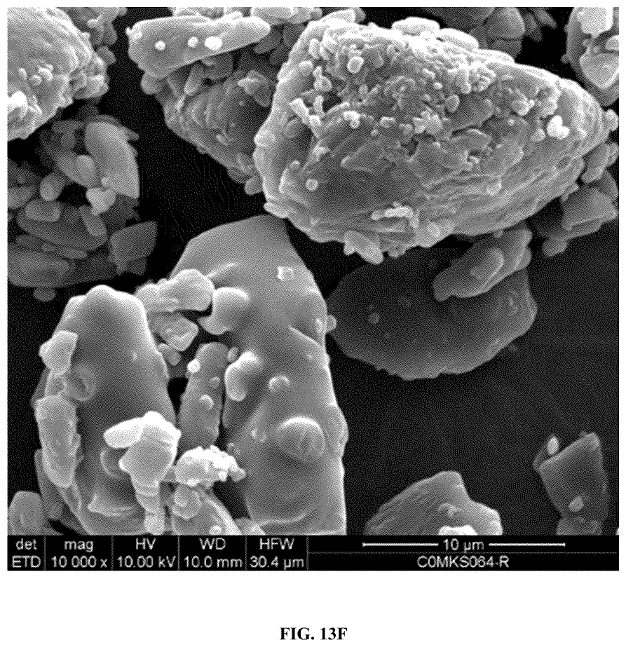

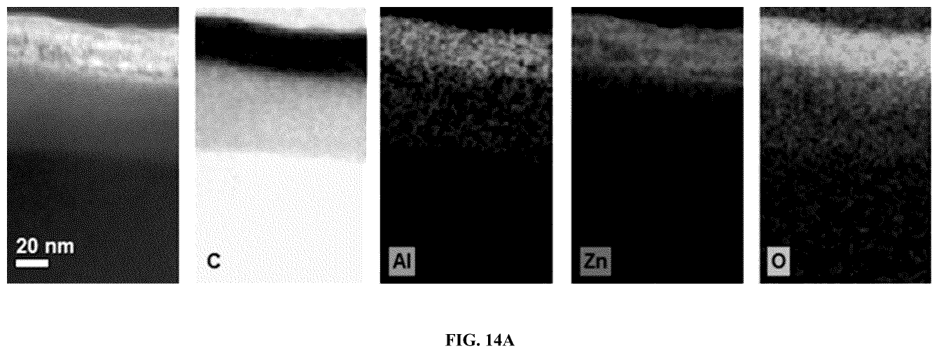

FIGS. 13A-13F show the SEM images of uncoated and coated particles (AZO-2).

FIGS. 13A-13C show SEM images of uncoated particles. FIGS. 13D-13F show SEM images of coated particles. No significant change in morphology of the particles or particle surface was observed after the AZO coating process.

### Release Over Time (Dissolution) Analysis

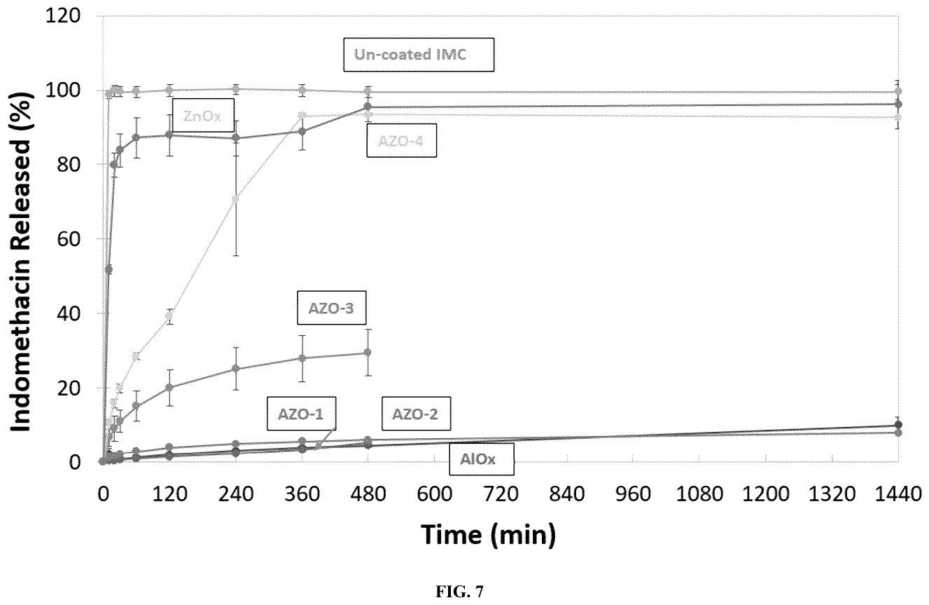

FIG. 7 depicts the results of a study of indomethacin dissolution in pH 7.2 sodium phosphate buffer solution in a USP2 dissolution apparatus (paddle) at 37° C. at 100 rpm stirring speed. As shown in the figure, both uncoated particles and particles coated with ZnOx exhibited an immediate indomethacin release profile. The AlOx coated particles exhibited minimal indomethacin release. By comparison, the AZO coated particles exhibited controlled indomethacin release with release % controlled by AZO oxide composition (Al/Zn ratio). Again, the indomethacin release rates for AZO coated particles were in between that of ZnOx coated particles and that of AlOx coated particles. The indomethacin release rate of AZO coated particles decreases as the Al/Zn ratio increases.

The results suggest that by varying the Al/Zn ratio in an AZO coating, the release rate of a drug from AZO coated particles can be adjusted.

### Particle Size Distribution Analysis

Table 2, below, shows the particle size distribution before and after the AZO coating process, as measured by laser diffraction. As shown in the table below, there is no significant change in particle size distribution after the coating process.

TABLE 2
Size distribution for coated particles

| Sample | D10 (μm) | D50 (μm) | D90 (μm) | Span |
|---|---|---|---|---|
| UC-m-IMC | 4.4 ± 0.4 | 13.3 ± 0.1 | 24.5 ± 0.5 | 1.5 |
| AZO-1 Coated Sample | 5.5 ± 0.6 | 13.4 ± 0.6 | 23.5 ± 0.7 | 1.4 |
| AZO-2 Coated Sample 1 | 5.8 ± 0.1 | 13.8 ± 0.3 | 25.2 ± 1.3 | 1.4 |
| AZO-3 Coated Sample | 3.2 ± 0.3 | 11.6 ± 0.1 | 21.2 ± 0.6 | 1.5 |
| AZO-2 Coated Sample 2 | 5.0 ± 0.6 | 12.6 ± 0.6 | 21.6 ± 0.7 | 1.3 |
| AZO-2 Coated Sample 3 | 5.0 ± 0.1 | 13.6 ± 0.3 | 25.8 ± 1.1 | 1.5 |
| AlOx Coated Sample 1 | 5.2 ± 0.1 | 13.4 ± 0.3 | 25.2 ± 0.9 | 1.5 |
| AlOx Coated Sample 2 | 3.7 ± 0.4 | 11.0 ± 0.02 | 20.2 ± 0.6 | 1.5 |

### X-Ray Diffraction (XRD) Analysis

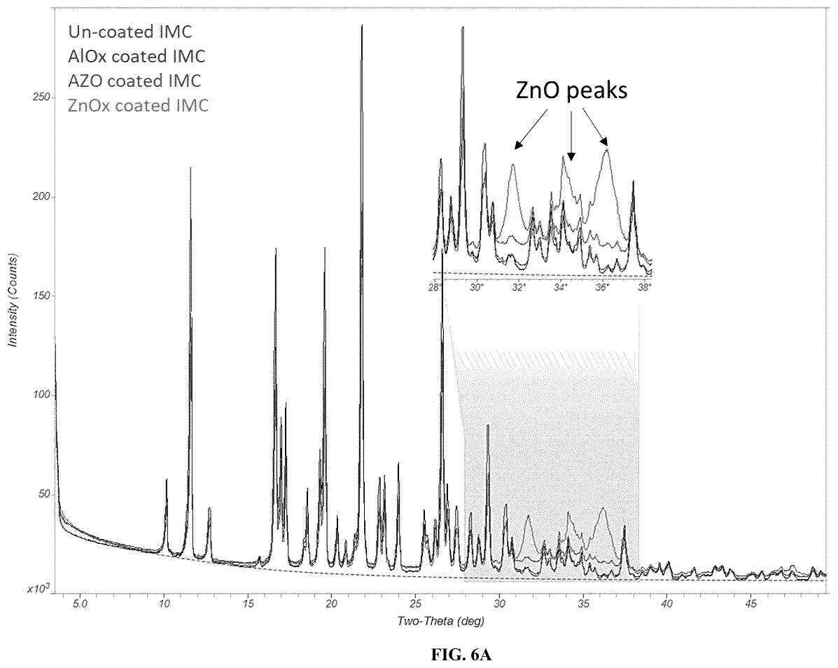

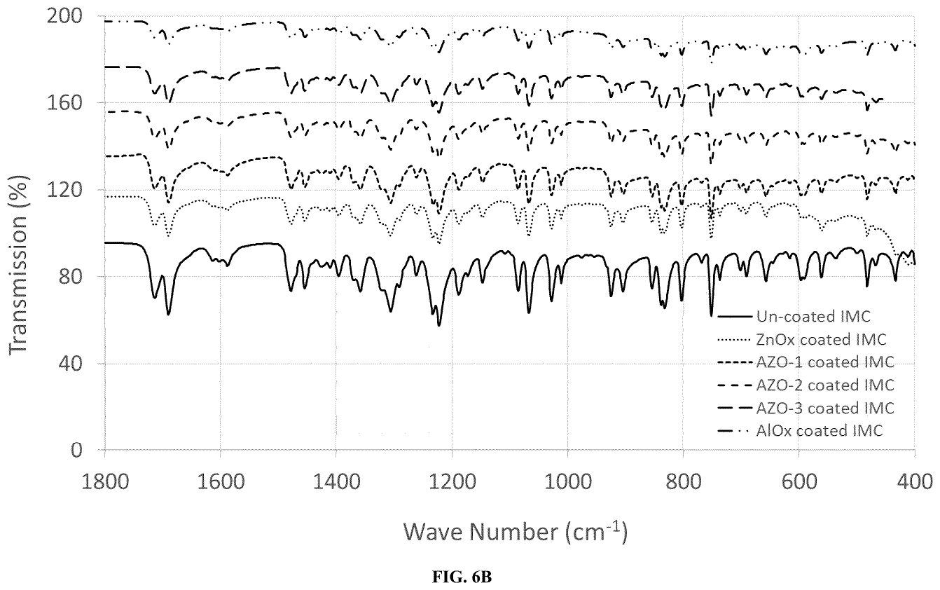

FIG. 6A shows the XRD analysis results before and after applying an AZO-2 coating to indomethacin particles. As shown in the figure, there was no change in XRD signals of the y polymorph of indomethacin after the coating process, indicating that there was no structural change in the indomethacin after the coating process, and that the indomethacin was not damaged by the coating process. The AlOx and AZO-2 coating are amorphous, with a low intensity broad hump at 28-38° 2θ for the AZO coating and no appreciable XRD signals for the amorphous AlOx coating due to its low content in the sample. For the ZnOx coated sample, XRD peaks for crystalline ZnOx were observed (see the zoomed-in XRD patterns).

### Fourier Transform Infrared (FTIR) Analysis

FIG. 6B shows FTIR analysis results before and after applying an AZO coating to indomethacin. As shown in the figure, there was no significant change in FTIR spectra after the coating process, indicating that there was no chemical change in the indomethacin after the coating process, and that the indomethacin was not damaged by the coating process. The absorption peaks for the coating oxide are weak except for ZnOx which is seen as the broad peak at 500-400 cm-1 (with peaks from indomethacin superimposed on it).

### Release Over Time (Dissolution) Analysis

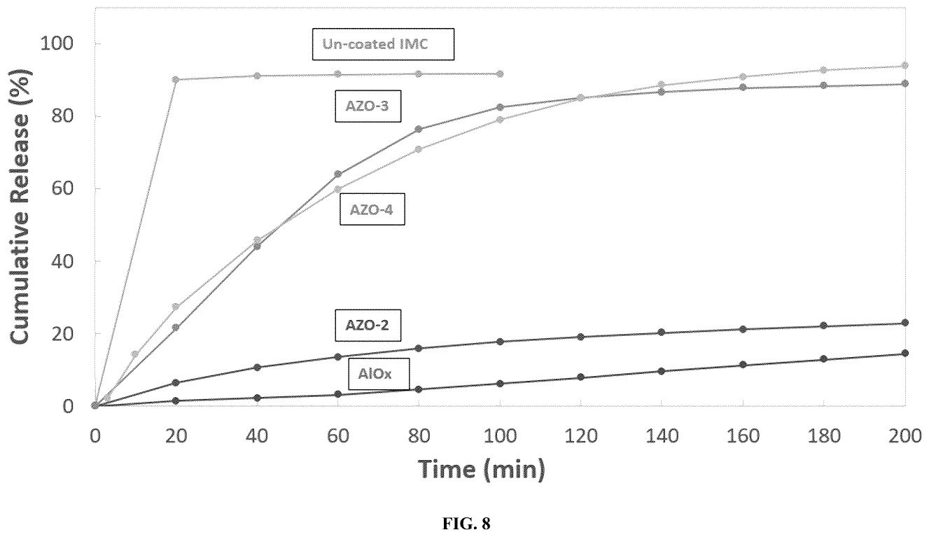

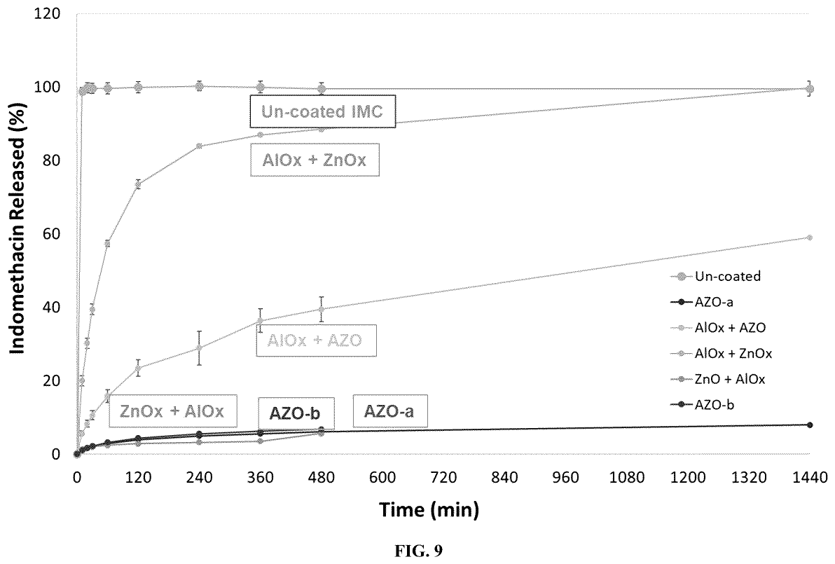

FIG. 8 depicts the results of a study of indomethacin dissolution in pH 7.2 PIPES buffer solution in a USP4 dissolution apparatus (flow through) at 37° C. at a 16 ml/min flow rate. As shown in the figure, uncoated particles exhibited an immediate indomethacin release (fast release) profile. The AlOx coated particles exhibited minimal indomethacin release. By comparison, the AZO coated particles exhibited controlled indomethacin release with release % controlled by AZO oxide composition (Al/Zn ratio). Again, the indomethacin release rates for AZO coated particles were in between that of ZnOx coated particles and that of AlOx coated particles. The release rate of AZO coated particles decreases as the Al/Zn ratio increases.

### Example 2: Aluminum Zinc Oxide Coating with Different Structures and Similar Al/Zn Ratio

Different coating structures of aluminum zinc oxide coating with an overall Al/Zn mass ratio ˜0.3 were deposited on micronized indomethacin powder (D50 of 5 μm). The structures of the coating are listed in the table below. The coatings include: an AZO coating layer; a two-layer coating with an inner AlOx layer and an outer AZO layer; a two-layer coating with an inner AlOx layer and an outer ZnOx layer; a two-layer coating with an inner ZnOx layer and an outer AZO layer; a two-layer coating with an inner ZnOx layer and an outer AlOx layer. The AZO layers were generated using the supercycle process. The AlOx layers and the ZnOx layers were generated using TMA and H₂O process and DEZ and H₂O process, respectively. Control AlOx-coated and ZnOx-coated particles were also created. In general, the operating temperature was 50° C.; the TMA, DEZ and H₂O pressures in the process were 2 Torr; and the purging nitrogen pressure was 8 torr.

TABLE 3
Different coating structures of aluminum zinc oxide coating

| | | Al/Zn | | Oxide |
|---|---|---|---|---|
| | | (wt | Oxide wt % | wt % |
| Sample | Coating Structure | ratio) | (ICP-OES) | (TGA) |
| AZO-a | AZO (Al first) | 0.31 | 15.4 | 14.4 |
| AIOx + | AlOx inner ± AZO (lower | 0.40 | 15.4 | 15.4 |
| AZO | Al AZO) outer | | | |
| AIOx + | AlOx inner + ZnOx outer | 0.27 | 15.0 | 13.9 |
| ZnOx | | | | |
| ZnOx + | ZnOx inner + AlOx outer | 0.27 | 14.1 | 13.3 |
| AIOx | | | | |
| ZnOx + | ZnOx inner + AZO (higher | 0.24 | 14.4 | 14.3 |
| AZO | Al AZO) outer | | | |
| AZO-b | AZO (Zn first) | 0.32 | 14.5 | 14.2 |

To evaluate the properties of the coated particles, the coated particles were subjected to various analyses.

### Thermogravimetric Analysis (TGA) Analysis

Table 3 shows the TGA analysis results of the coated particles. As shown in the table, the total inorganic material (oxide wt %) constitutes about 13-15% of the coated particles.

### Al/Zn Ratio (Wt %) Analysis

Table 3 also shows the ratio between aluminum and zinc in the coated particles.

### Transmission Electron Microscopy (TEM) Analysis

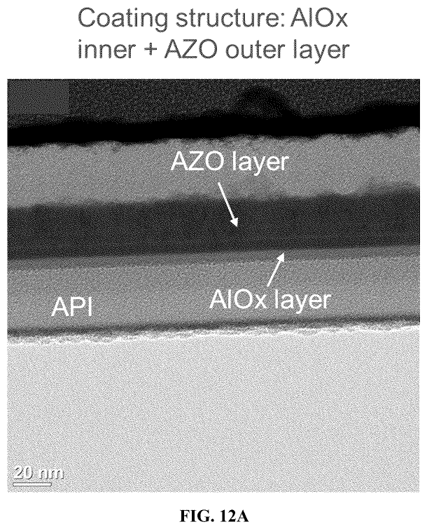

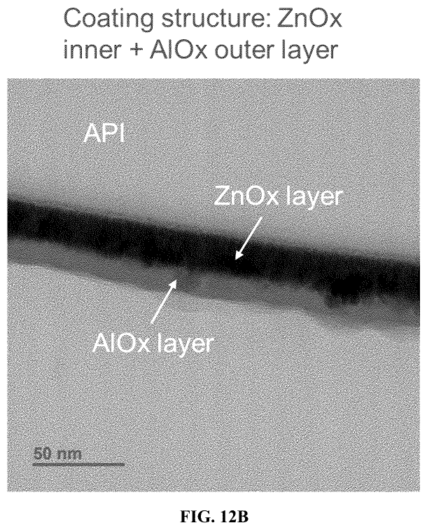

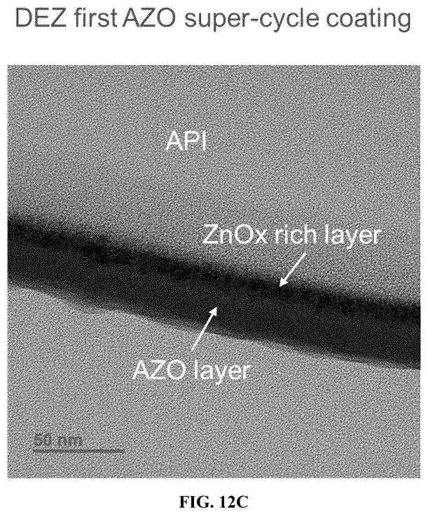

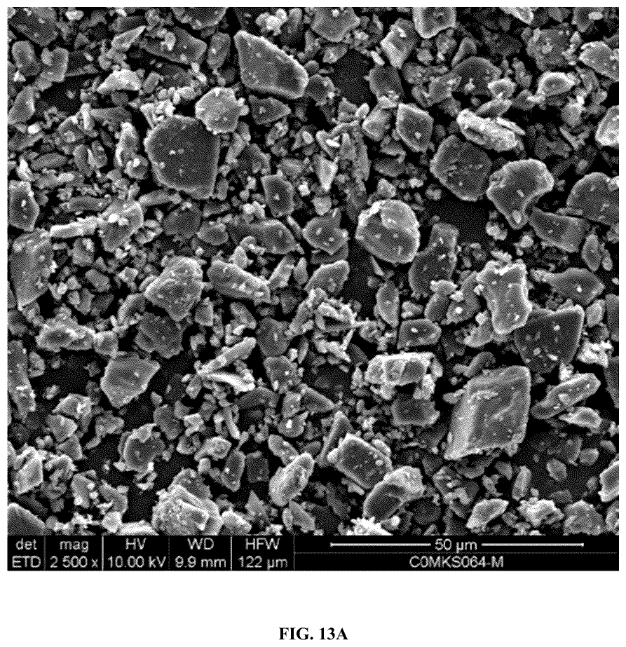

FIGS. 12A-12C show the TEM images of the coated particles described in Table 3.

FIG. 12A shows a TEM image of an AlOx+AZO coated particle described in Table 3. Distinctive AlOx and AZO layers were observed.

FIG. 12B shows a TEM image of a ZnOx+AlOx coated particle described in Table 3. Distinctive ZnOx and AlOx layers were observed.

FIG. 12C shows a TEM image of AZO-b (DEZ first) coated particle described in Table 3. A thin ZnOx rich layer at the indomethacin-coating interface was observed.

### Release Over Time (Dissolution) Analysis

FIG. 9 depicts the result of a study of indomethacin dissolution in pH 7.2 sodium phosphate buffer solution in a USP2 dissolution apparatus (paddle) at 37° C. at 100 rpm stirring speed for the coated particles described in Table 3. As shown in the figure, uncoated particles exhibited an immediate indomethacin release profile. By comparison, the coated particles exhibited controlled indomethacin release.

### Example 3: Micronized Indomethacin Particles Coated with an AlOx Layer

Indomethacin particles were coated with an AlOx layer. The thickness of the layer varied. The coating oxide content and estimated thicknesses as shown in the table below.

TABLE 4
AlOx coated particles

| | Oxide wt. % | Thickness |
|---|---|---|
| Sample ID | (TGA) | (nm; from TGA) |
| UC-m-IMC | n/a | n/a |
| AlOx thick coating | 10.7 | 22.0 |
| AlOx medium 2 coating | 8.94 | 18.4 |
| AlOx medium 1 coating | 7.06 | 14.5 |
| AlOx thin coating | 4.91 | 10.1 |

To evaluate the properties of the coated particles, the coated particles described in Table 4 were subjected to various analyses.

### Thermogravimetric Analysis (TGA) Analysis

Table 4 shows the TGA analysis results of the coated particles. As shown in Table 4, the total inorganic material (oxide wt %) constitutes about 5-11% of the coated particles.

### Thickness Analysis

Based on the TGA analysis, Table 4 also shows estimated thickness of the coating on the particles.

### Release Over Time (Dissolution) Analysis

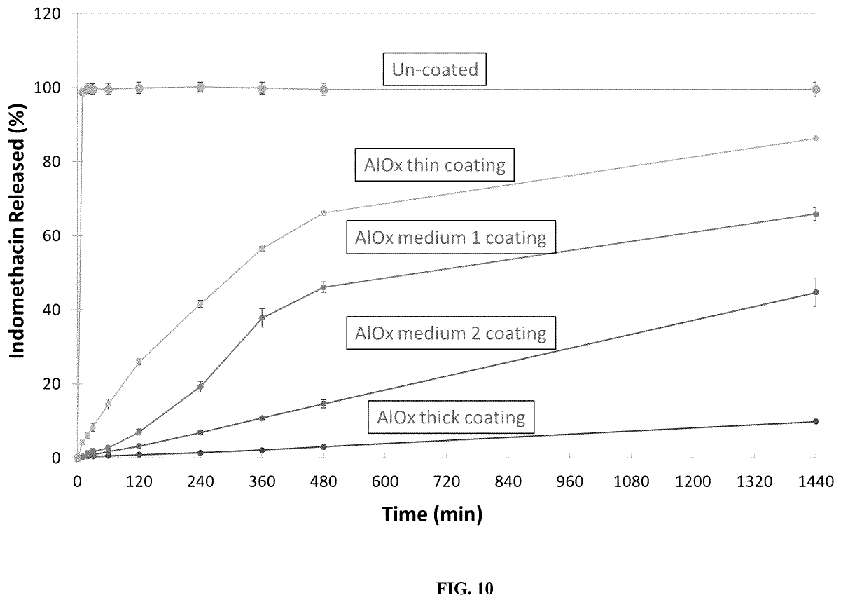

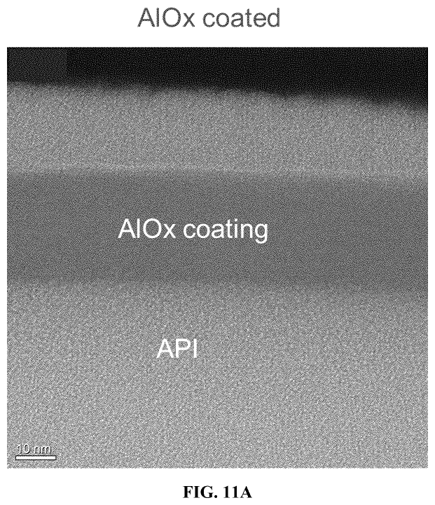

FIG. 10 depicts the results of a study or indomethacin dissolution in pH 7.2 sodium phosphate buffer solution in a USP2 dissolution apparatus (paddle) at 37° C. at 100 rpm stirring speed. As shown in the figure, uncoated particles exhibited an immediate indomethacin release profile. By comparison, the AlOx coated particles exhibited controlled indomethacin release. The release rate of the AlOx coated particles decreased as the thickness of the AlOx coating increased.

### Example 4: Micronized Indomethacin Particles Coated with AZO Coating (Al/Zn 1:3) of Various Thicknesses

A supercycle process was applied to uncoated particles (drug-containing core) following the coating methods in Example 1 above for AZO-2 composition. Different coating thicknesses were deposited by adjusting the number of supercycles.

To evaluate the properties of the coated particles, the coated particles were subjected to various analyses. The release rate of drug from AZO coated particles generally decreases as the AZO coating thickness increases.

### Transmission Electron Microscopy (TEM) and Electron Energy Loss Spectroscopy (EELS) Analysis

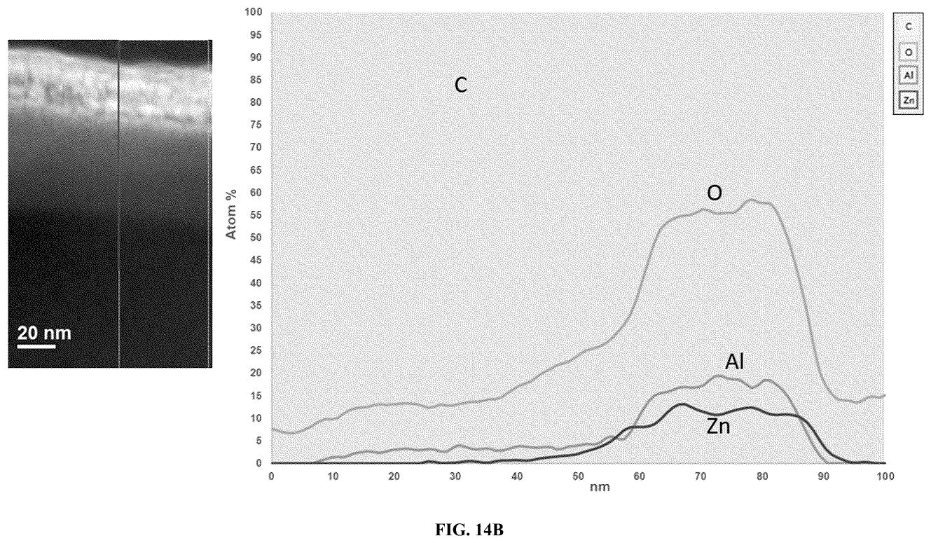

FIGS. 14A-14B show TEM/EELS elemental mapping of the AZO thick coating (AZO-2). In FIG. 14A, the first panel on the left shows the TEM image of the area being mapped, and other panels show the EELS mapping results of C, Al, Zn, O, Cl, and N. The results show relatively homogeneous distribution of Al and Zn across the thickness of the AZO coating.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of preparing coated particles comprising drug-containing core enclosed by an inorganic oxide coating, wherein the drug-containing core comprises a drug, the method comprising the sequential steps of:
(a) loading particles comprising a drug into a chamber of a reactor;
(b) performing a first number of first cycles, wherein each first cycle comprises steps (b1)-(b4):
(b1) applying a vaporous or gaseous first inorganic oxide precursor to the particles in the reactor by pulsing the vaporous or gaseous first inorganic oxide precursor into the reactor;
(b2) purging using an inert gas or performing one or more pump-purge cycles of the reactor using an inert gas;
(b3) applying a vaporous or gaseous oxidant to the particles in the reactor by pulsing the oxidant into the reactor;
(b4) purging using an inert gas or performing one or more pump-purge cycles of the reactor using an inert gas;
(c) performing a second number of second cycles, wherein each second cycle comprises steps (c1)-(c4):
(c1) applying a vaporous or gaseous second inorganic oxide precursor to the particles in the reactor by pulsing the vaporous or gaseous second inorganic oxide precursor into the reactor;
(c2) purging using an inert gas or performing one or more pump-purge cycles of the reactor using an inert gas;
(c3) applying a vaporous or gaseous oxidant to the particles in the reactor by pulsing the oxidant into the reactor;
(c4) purging using an inert gas or performing one or more pump-purge cycles of the reactor using an inert gas; and
(d) repeating steps (b)-(c) at least once;
wherein, the first inorganic oxide precursor and second inorganic oxide precursor are different; in each repeat of step (b) the first number is independently selected from 1-10 or 1-20; and in each repeat of step (c) the second number is independently selected from 1-10 or 1-20.

2.-5. (canceled)

6. The method of claim 1, wherein the inorganic oxide precursors are selected from the group consisting of a zinc oxide precursor, an aluminum oxide precursor and a silicon oxide precursor.

7. The method of claim 1, wherein each of the first and second inorganic oxide precursor is selected from DEZ and TMA and either: a) the first inorganic oxide precursor is TMA and the second inorganic oxide precursor is DEZ; or b) the first inorganic oxide precursor is DEZ and the second inorganic oxide precursor is TMA.

8. The method of claim 1, wherein each repeat of step (b) the first number is the same and selected from 1-10; and in each repeat of step (c) the second number is the same and selected from 1-10.

9.-13. (canceled)

14. The method of claim 1, wherein each of steps (b1), (b3), (c1) and (c3) comprises: (i) introducing the vaporous or gaseous inorganic oxide precursor into the chamber, (ii) allowing a holding time to pass, and (iii) pumping the vaporous or gaseous inorganic oxide precursor of the chamber; and repeating steps (i)-(ii) at least once.

15.-17. (canceled)

18. The method of claim 1, wherein the first cycles and second cycles take place at a temperature between 25° C. and 60° C.

19.-21. (canceled)

22. The method of claim 1, wherein each pump-purge cycle comprises flowing the inert gas into the reactor chamber to a desired pressure and after a delay time pumping the inert gas out of the reactor until the pressure of the inert gas is below 1 torr and repeating the steps of flowing the inert gas into the reactor chamber to a desired pressure and after a delay time pumping the inert gas out of the reactor until the pressure of the inert gas is below 1 torr.

23. The method of claim 1, wherein: a) the first and second inorganic acid precursors are selected from an aluminum oxide precursor and a zinc oxide precursor; b) the first and second inorganic acid precursors are selected from an aluminum oxide precursor and a silicone oxide precursor; or c) the first and second inorganic acid precursors are selected from an silicone oxide precursor and a zinc oxide precursor.

24.-26. (canceled)

27. The method of claim 1, the method further comprising, after step (d):
(e) performing a third number of third cycles, wherein each third cycle comprises steps (e1)-(e4):
(e1) applying a vaporous or gaseous third inorganic oxide precursor to the particles in the reactor by pulsing the vaporous or gaseous third precursor into the reactor;
(e2) purging using an inert gas or performing one or more pump-purge cycles of the reactor using an inert gas;
(e3) applying a vaporous or gaseous oxidant to the particles in the reactor by pulsing the oxidant into the reactor;
(e4) purging using an inert gas or performing one or more pump-purge cycles of the reactor using an inert gas;
(f) performing a fourth number of fourth cycles, wherein each fourth cycle comprises steps (f1)-(f4):
(f1) applying a vaporous or gaseous fourth inorganic oxide precursor to the particles in the reactor by pulsing the vaporous or gaseous fourth precursor into the reactor;
(f2) purging using an inert gas or performing one or more pump-purge cycles of the reactor using an inert gas;
(f3) applying a vaporous or gaseous oxidant to the particles in the reactor by pulsing the oxidant into the reactor;
(f4) purging using an inert gas or performing one or more pump-purge cycles of the reactor using an inert gas; and
(g) repeating steps (e)-(f) at least once,
wherein, the third and fourth precursor are different; in each repeat of step (e) the third number is independently selected from 1-10 or 1-20; and in each repeat of step (f) the fourth number is independently selected from 1-10 or 1-20.

28.-31. (canceled)

32. The method of claim 1, wherein the coating constitutes 1-20% wt/wt of the coated particles.

33.-36. (canceled)

37. A coated particle prepared by the method of claim 1.

38. (canceled)

39. A coated particle comprising drug-containing core enclosed by an inorganic oxide coating, wherein the drug-containing core comprises a drug, and the inorganic oxide coating comprising at least one layer composed of three elements (“three element inorganic oxide layer”), wherein the three elements are: a) aluminum, zinc and oxygen; b) aluminum, silicon and oxygen; or c) silicon, zinc and oxygen.

40. The coated particle of claim 39, wherein the three element inorganic oxide layer is at least 2 nm thick.

41. The coated particle of claim 39, wherein the three element inorganic oxide layer is 2-50 nm thick.

42. The coated particle of claim 39, wherein the coated drug particle is 5-30% by weight inorganic oxide.

43. The coated particle of claim 39, wherein the three element inorganic oxide layer is composed of: a) aluminum, zinc and oxygen and the ratio of aluminum to zinc varies in the layer; b) aluminum, silicone and oxygen and the ratio of aluminum to silicon varies in the layer; or c) silicon, zinc and oxygen and the ratio of silicon to zinc varies in the layer.

44. The coated particle of claim 39, wherein the inorganic oxide coating further comprises at least one inorganic oxide coating layer composed of two elements (“two element inorganic oxide coating layer), wherein the two elements are: a) zinc and oxygen; b) aluminum and oxygen; or c) silicon and oxygen.

45. The coated particle of claim 44, wherein the two element inorganic oxide layer is at least 1 nm thick.

46.-48. (canceled)

49. A pharmaceutical composition comprising the coated particle of claim 39 and a pharmaceutically acceptable excipient or carrier.

50. A pharmaceutical composition comprising the coated particle of claim 39 and water.

51. The method of claim 1, further comprising:
i. applying a vaporous or gaseous inorganic oxide precursor to the particles in the reactor by pulsing the vaporous or gaseous inorganic oxide precursor into the reactor;
ii. purging using an inert gas or performing one or more pump-purge cycles of the reactor using an inert gas;
iii. applying a vaporous or gaseous oxidant to the particles in the reactor by pulsing the oxidant into the reactor;
iv. purging using an inert gas or performing one or more pump-purge cycles of the reactor using an inert gas;
repeating steps i.-iv. at least once, wherein steps i.-iv. can take place: (1) between steps a) and b); 2) between steps d) and e); or after step g).

52. The method of claim 51, wherein the first inorganic oxide precursor is an aluminum oxide precursor, a zinc oxide precursor or a silicon oxide precursor.

53.-55. (canceled)
